(19) United States
(12) Patent Application Publication
(10) Pub. No.: US 2005/0267032 A1
(43) Pub. Date: Dec. 1, 2005

(54) Sulfone-containing prodrugs

(75) Inventors: Alan Bradley Fulp, Willow Springs, NC (US); Grant Andrew McNaughton-Smith, Morrisville, NC (US); Aimee D. Reed, Willoughby, OH (US); Xiadong Wang, Chapel Hill, NC (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Icagen, Inc., Durham, NC (US)

(21) Appl. No.: 11/136,243

(22) Filed: May 23, 2005

Related U.S. Application Data
(60) Provisional application No. 60/573,233, filed on May 21, 2004.

(51) Int. Cl. A61K 38/18; A61K 31/381

(52) U.S. Cl. 514/12; 514/79; 514/447; 514/575; 549/5; 549/76

(57) ABSTRACT

Compounds, compositions and methods are provided which are useful in the treatment of diseases through the modulation of potassium ion flux through voltage-dependent potassium channels. More particularly, the invention provides sulfone-containing prodrugs, and compositions and methods utilizing sulfone-containing prodrugs that are useful in the treatment of diseases by blocking potassium channels associated with the onset or recurrence of the indicated conditions. Exemplary diseases treatable with the compounds, compositions and methods of the invention include sickle cell disease and glaucoma.

### CROSS REFERENCE TO RELATED APPLICATION

This application claims the benefit of U.S. Provisional Patent Application No. 60/573,233, filed May 21, 2004, which is herein incorporated by reference in its entirety for all purposes.

### BACKGROUND OF THE INVENTION

Ion channels are cellular proteins that regulate the flow of ions, including calcium, potassium, sodium and chloride into and out of cells. These channels are present in all human cells and affect such physiological processes as nerve transmission, muscle contraction, cellular secretion, regulation of heartbeat, dilation of arteries, release of insulin, and regulation of renal electrolyte transport. Among the ion channels, potassium channels are the most ubiquitous and diverse, being found in a variety of animal cells such as nervous, muscular, glandular, immune, reproductive, and epithelial tissue. These channels allow the flow of potassium in and/or out of the cell under certain conditions. For example, the outward flow of potassium ions upon opening of these channels makes the interior of the cell more negative, counteracting depolarizing voltages applied to the cell. These channels are regulated, e.g., by calcium sensitivity, voltage-gating, second messengers, extracellular ligands, and ATP-sensitivity.

Potassium channels are made by alpha subunits that fall into at least 8 families, based on predicted structural and functional similarities (Wei et al., Neuropharmacology 35(7): 805-829 (1997)). Three of these families (Kv, eag-related, and KQT) share a common motif of six transmembrane domains and are primarily gated by voltage. Two other families also contain this motif but are gated by cyclic nucleotides (CNG) and calcium (small conductance and intermediate conductance potassium channels), respectively. The small conductance and intermediate conductance calcium activated potassium channels comprise a family of calcium activated potassium channels gated solely by calcium, with a unit conductance of 2-20 and 20-85 pS, respectively. Macroscopic and unitary intermediate conductance calcium activated potassium channel currents show inward rectification (see, e.g., Ishii et al., Proc. Natl. Acad. Sci USA 94: 11651-11656 (1997). The three other families of potassium channel alpha subunits have distinct patterns of transmembrane domains. Slo family potassium channels, or BK channels have seven transmembrane domains (Meera et al., Proc. Natl. Acad. Sci. U.S.A. 94(25): 14066-71 (1997)) and are gated by both voltage and calcium or pH (Schreiber et al., J. Biol. Chem. 273: 3509-16 (1998)). Another family, the inward rectifier potassium channels (Kir), belongs to a structural family containing two transmembrane domains, and an eighth functionally diverse family (TP, or “two-pore”) contains two tandem repeats of this inward rectifier motif.

Potassium channels are typically formed by four alpha subunits, and can be homomeric (made of identical alpha subunits) or heteromeric (made of two or more distinct types of alpha subunits). In addition, potassium channels made from Kv, KQT and Slo or BK subunits have often been found to contain additional, structurally distinct auxiliary, or beta, subunits. These subunits do not form potassium channels themselves, but instead they act as auxiliary subunits to modify the functional properties of channels formed by alpha subunits. For example, the Kv beta subunits are cytoplasmic and are known to increase the surface expression of Kv channels and/or modify inactivation kinetics of the channel (Heinemann et al., J. Physiol. 493: 625-633 (1996); Shi et al., Neuron 16(4): 843-852 (1996)). In another example, the KQT family beta subunit, minK, primarily changes activation kinetics (Sanguinetti et al., Nature 384: 80-83 (1996)).

The intermediate conductance, calcium activated potassium channel is also called SK4, KCa4, IKCa, SMIK, and Gardos. Intermediate conductance, calcium activated potassium channels have been previously described in the literature by their electrophysiology. For example, the Gardos channel, a well-known intermediate conductance, calcium activated potassium channel, is opened by submicromolar concentrations of internal calcium and has a rectifying unit conductance, ranging from 50 pS at −120 mV to 13 pS at 120 mV (symmetrical 120 mM K+; Christopherson, J. Membrane Biol. 119: 75-83 (1991)). Intermediate conductance, calcium activated potassium channels are blocked by charybdotoxin (CTX) but not the structurally related peptide iberiotoxin (IBX), both of which block BK channels (Brugnara et al., J. Membr Biol. 147: 71-82 (1995)). Intermediate conductance, calcium activated potassium channels are also blocked by maurotoxin. Apamin, a potent blocker of certain native (Vincent et al., J. Biochem. 14: 2521 (1975); Blatz & Magleby, Nature 323: 718-720 (1986)) and cloned SK channels does not block intermediate conductance, calcium activated potassium channels (de-Allie et al., Br. J. Pharm. 117: 479-487 (1996)). The Gardos channel is also blocked by some imidazole compounds, such as clotrimazole, but not ketoconazole (Brugnara et al, J. Clin. Invest., 92: 520-526 (1993)). Intermediate conductance, calcium activated potassium channels can therefore be distinguished from the other calcium activated potassium channels by their biophysical and pharmacological profiles. Intermediate conductance, calcium activated potassium channels from different tissues have been reported to possess a wide range of unit conductance values.

Human intermediate conductance, calcium activated potassium channels have been cloned and characterized (see, e.g., Ishii et al., Proc. Natl. Acad. Sci. USA 94: 11651-11656 (1997); Genbank Accession No. AF0225150; Joiner et al., Proc. Natl. Acad. Sci. USA 94: 11013-11018 (1997); Genbank Accession No. AF000972; Lodsdon et al., J. Biol. Chem. 272: 32723-32726 (1997); Genbank Accession No. AF022797; and Jensen et al., Am. J. Physiol. 275: C848-856 (1998); see also WO 98/11139; WO 99/03882; WO 99/25347; and WO 00/12711). Non-human intermediate conductance, calcium activated potassium channels have also been cloned, e.g., from mouse and rats (see, e.g., Vandorpe et al., J. Biol. Chem. 273: 21542-21553 (1998); Genbank Accession No. NM_{—}032397; Warth et al., Pflugers Arch. 438: 437-444 (1999); Genbank Accession No. AJ133438; and Neylon et al., Circ. Res. (online)85: E33-E43 (1999); Genbank Accession No. AF190458). The gene for the intermediate conductance, calcium activated potassium channels is named KCNN4 and it is located on chromosome 19q13.2 (Ghanshani et al., Genomics 51: 160-161 (1998)).

The intermediate conductance, calcium activated potassium channel is implicated in the regulation of mammalian cell proliferation (see, for example, Wulff et al., Proc. Nat. Acad. Sci. USA 97: 8151-8156 (2000)), the dehydration and sickling of erythrocytes in sickle cell disease, among others (See, infra). Sickle cell anemia and the existence of sickle hemoglobin (Hb S) was the first genetic disease to be understood at the molecular level. Today, it is recognized that sickle cell disorders are be caused by inheritance of a sickle cell gene, such as β^{6Glu→Val} (Hemoglobin S), β^{6Glu→Lys} (Hemoglobin C), β2^{6Glu→Val} (Hemoglobin E), β^{98Val→Met} (Hemoglobin Köln), β^{99Asp→His} (Hemoglobin Yakima), β^{102Asn→Lys} (Hemoglobin Kansas), or combinations thereof. Current treatments for sickle cell disorders include administration of compounds such as antisickling agents (e.g. hydroxyurea), erythropoietin, and/or antibiotics (e.g. ceftriaxone and erythromycin).

Normal erythrocytes are comprised of approximately 70% water. Water crosses a normal erythrocyte membrane in milliseconds. Loss of cell water causes an exponential increase in cytoplasmic viscosity as the mean cell hemoglobin concentration (MCHC) rises above about 32 g/dl. Since cytoplasmic viscosity is a major determinate of erythrocyte deformability and sickling, the dehydration of the erythrocyte has substantial rheological and pathological consequences. Regulation of erythrocyte dehydration is recognized as an important therapeutic approach for treating sickle cell disease. Since cell water follows any osmotic change in intracellular ion concentration, maintaining the red cell's potassium concentration is of particular importance (Stuart et al., Brit J. Haematol. 69: 1-4 (1988)).

An approach towards therapeutically treating dehydrated sickle cells involves altering erythrocyte potassium flux by targeting a calcium-dependent potassium channel. In vitro studies have shown that clotrimazole, an imidazole-containing antimycotic agent, blocks Ca²⁺-activated K⁺ flux and cell dehydration in sickle erythrocytes (Brugnara et al., J. Clin. Invest. 92: 520-526 (1993)). Studies in a transgenic mouse model for sickle cell disease, SAD-1 mouse (Trudel et al., EMBO J. 11: 3157-3165 (1991)), show that oral administration of clotrimazole leads to inhibition of the red cell Gardos channel, increased red cell K⁺ content, a decreased mean corpuscular hemoglobin concentration (MCHC) and decreased cell density (De Franceschi et al., J. Clin. Invest. 93: 1670-1676 (1994)). Moreover, therapy with oral clotrimazole induces inhibition of the Gardos channel and reduces erythrocyte dehydration in patients with sickle cell disease (Brugnara et al., J. Clin. Invest. 97: 1227-1234 (1996)). Other antimycotic agents, which inhibit the Gardos channel in vitro, include miconazole, econazole butoconazole, oxiconazole and sulconazole (U.S. Pat. No. 5,273,992 to Brugnara et al.). All of these compounds contain an imidazole-like ring. i.e., a heteroaryl ring containing two or more nitrogens.

Although of demonstrable efficacy, the imidazole-based Gardos channel inhibitors that have been explored to date are hampered by several shortcomings including a well-documented potential for hepatotoxicity. This toxicity is exacerbated by the inhibitors' low potencies, non-specific interactions with potassium channels other than the Gardos channel and low bioavailabilities, each of which motivate for the administration of higher and more frequent dosages of the inhibitors.

Glaucoma is a disease characterized by increased intraocular pressure. Increased intraocular pressure is associated with many diseases including, but not limited to, primary open-angle glaucoma, normal tension glaucoma, angle-closure glaucoma, acute glaucoma, pigmentary glaucoma, neovascular glaucoma, or trauma related glaucoma, Sturge-Weber syndrome, uveitis, and exfoliation syndrome. By modulating these IK1 channels with potent and selective compounds, novel methods of lowering intraocular pressure may be found.

Currently, there are a variety of drugs available that employ different mechanisms to lower intraocular pressure, e.g., timolol, betaxolol, levobunolol, acetazolamide, methazolamide, dichlorphenamide, dorzolamide, brinzolamide, latanoprost, brimonidine, and rescula (see, e.g., U.S. Pat. No. 6,172,054, U.S. Pat. No. 6,172,109, and U.S. Pat. No. 5,652,236). Miotics, beta blockers, alpha-2 agonists, carbonic anhydrase inhibitors, beta adrenergic blockers, prostaglandins and docosanoid are all currently used alone or in combination to treat glaucoma. Miotics and prostaglandins are believed to lower intraocular pressure by increasing drainage of the intraocular fluid, while beta blockers, alpha-2 agonists and carbonic anhydrase are believed to lower intraocular pressure by decreasing production of intraocular fluid thereby reducing the flow of fluid into the eye. All are characterized by side effects ranging from red eye and blurring of vision to decreased blood pressure and breathing difficulties. Other, newer agents are also described in the patent literature including a class of sulfonamides described in WO 04/016221 and certain triarylmethane compounds described in WO 03/074038.

In view of the above-described shortcomings of currently known methods of treating diseases in which the intermediate conductance, calcium activated potassium channel is implicated, a substantial advance in the treatment of diseases related to potassium flux is expected from the discovery of new intermediate conductance, calcium activated potassium channel inhibitors. The present invention provides a new prodrug genus of such ion channel inhibitors based on a sulfone-containing scaffold.

### BRIEF SUMMARY OF THE INVENTION

The present invention provides sulfone-containing prodrug compounds capable of inhibiting the IK1 potassium channel, including the Gardos channel of erythrocytes, after undergoing a chemical alteration. The sulfone-containing prodrugs include a prodrug modification group, which is capable of being chemically altered (e.g. partially or completely cleaved) by an enzyme thereby forming an active IK1 potassium channel blocker. Blockade of the IK1 channel is a powerful therapeutic approach towards the treatment and/or prevention of diseases in which the IK1 channel is implicated, such as sickle cell disease, glaucoma and other conditions as described herein or otherwise known in the art. Compounds capable of inhibiting the IK1 channel are highly desirable, and are an object of the present invention.

Thus, in one aspect, the present invention provides compounds according to Formula (I):

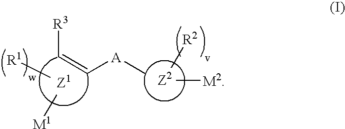

In Formula (I), ring system Z¹ is selected from aryl, and 5-membered heterocycloalkyl. Ring system Z² is selected from aryl, heteroaryl, (C₅-C₇)cycloalkyl, and 5 to 7 membered heterocycloalkyl. The symbols w and v are integers independently selected from0to4.

The symbols R¹ and R² represent moieties independently selected from —NH₂, —NO₂, —CF₃, —OCF₃, hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a fused ring, and OR⁹. R⁹ is selected from hydrogen, —CF₃, substituted or unsubstituted alkyl and substituted or unsubstituted heteroalkyl.

R³ is selected from —C(O)OR^{3A}, 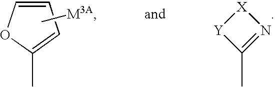
R^{3A} is selected from methyl, ethyl and —CF3. X is selected from —N═N—, —N═C(R^{3B})—, —C(R^{3B}R^{3C})—C(R^{3D}R^{3E}) and —C(R^{3B})═C(R^{3C}). R^{3B}, R^{3C}, R^{3D}, and R^{3E} are independently selected from hydrogen, substituted or unsubstituted lower alkyl, -M^{3B} and —CF₃. Y is selected from —O—, —N(M^{3C})— and —S—.

A is selected from -L¹-N(M⁴)-S(O)₂-L²-, and -L¹-S(O)_{q}-L²-. The symbol q is an integer selected from 0 to 2. L¹ and L² are independently selected from a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene.

M¹, M², M^{3A}, and M^{3B} are independently selected from hydrogen and -L⁵-X⁵—R⁵. L⁵ is independently selected from a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene. X⁵ is independently selected from —O— and —N(R⁶)—. R⁶ is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R⁵ is independently selected from C(O)—R^{5A} and 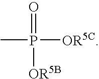
R^{5B} and R^{5C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R^{5A} is selected from -L^{5A1}-NR^{5A1}R^{5A2} and —OR^{5A3}. L^{5A1} is selected substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene. R^{5A1} and R^{5A3} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{5A2} is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, and —C(O)R^{5A5}. R^{5A5} is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

M^{3C} and M⁴ are independently selected from hydrogen, —CF₃, substituted or unsubstituted lower alkyl, —C(O)—R^{7A} and 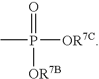
R^{7B} and R^{7C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R^{7A} is a selected from -L^{7A1}-NR^{7A1}R^{7A2}, and —OR^{7A3}. L^{7A1} is a selected from a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene. R^{7A1} and R^{7A3} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{7A2} is independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, and —C(O)R^{7A5}. R^{7A5} is a member selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In another aspect, the present invention provides pharmaceutical compositions comprising a pharmaceutically acceptable excipient and a compound of Formula (I).

Controlling diseases (e.g., sickle cell disease, glaucoma) via altering cellular ionic fluxes of cells affected by a disease is a powerful therapeutic approach. Moreover, basic understanding of the role of cellular ionic fluxes in both disease processes and normal physiology promises to provide new therapeutic modalities, regimens and agents. Compounds that alter cellular ion fluxes, particularly those that inhibit potassium flux, are highly desirable as both drugs and as probes for elucidating the basic mechanisms underlying these ion fluxes. Similarly, methods utilizing these compounds in basic research and in therapeutic applications are valuable tools in the arsenal of both the researcher and clinician. Therefore such compounds and methods are also an object of the present invention.

Thus, in a third aspect, the present invention provides a method of inhibiting potassium flux of a cell. The method comprises contacting a cell with an amount of a compound according to Formula (I), effective to inhibit the potassium flux.

An important therapeutic pathway for treatment of sickle cell disease is preventing, retarding, or reversing the dehydration of erythrocytes by manipulating the cellular ion fluxes of erythrocytes. Thus, in another aspect, the invention provides a method for reducing erythrocyte dehydration. The method comprises contacting an erythrocyte with an amount of a compound according to Formula (I), which is effective to reduce erythrocyte dehydration.

In a fifth aspect, the invention provides a method of treating or preventing sickle cell disease. The method comprises administering to a subject suffering sickle cell disease a therapeutically effective amount of a compound having a structure according to Formula (I).

In a sixth aspect, the present invention provides a method for reducing intraocular pressure. The method includes delivering to an eye, an amount of a compound according to Formula (I) sufficient to lower said intraocular pressure.

In a seventh aspect, the invention provides a method of treating or preventing glaucoma. The method comprises delivering to a subject suffering from or at risk of developing glaucoma a therapeutically effective amount of a compound according to Formula (I).

In another aspect, the invention is also directed to methods of treating or preventing mammalian cell proliferation. Thus, in another aspect, the invention provides methods of inhibiting mammalian cell proliferation as an approach towards the treatment or prevention of diseases characterized by unwanted or abnormal cell proliferation. In its broadest sense, these methods involve only a single step—the administration of an effective amount of at least one pharmacologically active compound according to the invention to a mammalian cell in situ. In exemplary embodiment, the compounds may act cytostatically, cytotoxically, or by a combination of both mechanisms to inhibit cell proliferation. Mammalian cells treatable in this manner include, e.g., vascular smooth muscle cells, fibroblasts, endothelial cells, various pre-cancer cells and various cancer cells. In a preferred embodiment, cell proliferation is inhibited in a subject suffering from a disorder that is characterized by unwanted or abnormal cell proliferation. Such diseases are described more fully below.

In an exemplary method of the invention, an effective amount of at least one compound according to the invention, or a pharmaceutical composition thereof, is administered to a patient suffering from a disorder that is characterized by abnormal cell proliferation. While not intending to be bound by any particular theory, it is believed that administration of an appropriate amount of a compound according to the invention to a subject inhibits cell proliferation by altering the ionic fluxes associated with early mitogenic signals. Such alteration of ionic fluxes is thought to be due to the ability of the compounds of the invention to inhibit potassium channels of cells. The method can be used prophylactically to prevent unwanted or abnormal cell proliferation, or may be used therapeutically to reduce or arrest proliferation of abnormally proliferating cells. The compound, or a pharmaceutical formulation thereof, can be applied locally to proliferating cells to arrest or inhibit proliferation at a desired time, or may be administered to a subject systemically to arrest or inhibit cell proliferation.

Diseases which are characterized by abnormal cell proliferation that can be treated or prevented by means of the present invention include, but are not limited to, blood vessel proliferative disorders, fibrotic disorders, atherosclerotic disorders and various cancers. Blood vessel proliferation disorders generally refer to angiogenic and vasculogenic disorders generally resulting in abnormal proliferation of blood vessels. The formation and spreading of blood vessels, or vasculogenesis and angiogenesis, respectively, play important roles in a variety of physiological processes such as embryonic development, corpus luteum formation, wound healing and organ regeneration. They also play a pivotal role in cancer development. Other examples of blood vessel proliferative disorders include arthritis, where new capillary blood vessels invade the joint and destroy cartilage and ocular diseases such as diabetic retinopathy, where new capillaries in the retina invade the vitreous, bleed and cause blindness and neovascular glaucoma.

Another example of abnormal neovascularization is that associated with solid tumors. It is now established that unrestricted growth of tumors is dependent upon angiogenesis and that induction of angiogenesis by liberation of angiogenic factors can be an important step in carcinogenesis. For example, basic fibroblast growth factor (bFGF) is liberated by several cancer cells and plays a crucial role in cancer angiogenesis. The demonstration that certain animal tumors regress when angiogenesis is inhibited has provided the most compelling evidence for the role of angiogenesis in tumor growth. Other cancers that are associated with neovascularization include hemangioendotheliomas, hemangiomas and Kaposi's sarcoma.

Proliferation of endothelial and vascular smooth muscle cells is the main feature of neovascularization. The invention is useful in inhibiting such proliferation, and therefore in inhibiting or arresting altogether the progression of the angiogenic condition which depends in whole or in part upon such neovascularization. The invention is particularly useful when the condition has an additional element of endothelial or vascular smooth muscle cell proliferation that is not necessarily associated with neovascularization. For example, psoriasis may additionally involve endothelial cell proliferation that is independent of the endothelial cell proliferation associated with neovascularization. Likewise, a solid tumor which requires neovascularization for continued growth may also be a tumor of endothelial or vascular smooth muscle cells. In this case, growth of the tumor cells themselves, as well as the neovascularization, is inhibited by the compounds described herein.

The invention is also useful for the treatment of fibrotic disorders such as fibrosis and other medical complications of fibrosis which result in whole or in part from the proliferation of fibroblasts. Medical conditions involving fibrosis (other than atherosclerosis, discussed below) include undesirable tissue adhesion resulting from surgery or injury.

Other cell proliferative disorders which can be treated by means of the invention include arteriosclerotic conditions. Arteriosclerosis is a term used to describe a thickening and hardening of the arterial wall. An arteriosclerotic condition as used herein means classical atherosclerosis, accelerated atherosclerosis, atherosclerotic lesions and any other arteriosclerotic conditions characterized by undesirable endothelial and/or vascular smooth muscle cell proliferation, including vascular complications of diabetes.

Proliferation of vascular smooth muscle cells is a main pathological feature in classical atherosclerosis. It is believed that liberation of growth factors from endothelial cells stimulates the proliferation of subintimal smooth muscle which, in turn, reduces the caliber and finally obstructs the artery. The invention is useful in inhibiting such proliferation, and therefore in delaying the onset of, inhibiting the progression of, or even halting the progression of such proliferation and the associated atherosclerotic condition.

Proliferation of vascular smooth muscle cells produces accelerated atherosclerosis, which is the main reason for failure of heart transplants that are not rejected. This proliferation is also believed to be mediated by growth factors, and can ultimately result in obstruction of the coronary arteries. The invention is useful in inhibiting such obstruction and reducing the risk of, or even preventing, such failures.

Vascular injury can also result in endothelial and vascular smooth muscle cell proliferation. The injury can be caused by any number of traumatic events or interventions, including vascular surgery and balloon angioplasty. Restenosis is the main complication of successful balloon angioplasty of the coronary arteries. It is believed to be caused by the release of growth factors as a result of mechanical injury to the endothelial cells lining the coronary arteries. Thus, by inhibiting unwanted endothelial and smooth muscle cell proliferation, the compounds described herein can be used to delay, or even avoid, the onset of restenosis.

Other atherosclerotic conditions which can be treated or prevented by means of the present invention include diseases of the arterial walls that involve proliferation of endothelial and/or vascular smooth muscle cells, such as complications of diabetes, diabetic glomerulosclerosis and diabetic retinopathy.

The compounds described herein are also useful in treating or preventing various types of cancers. Cancers which can be treated by means of the present invention include, but are not limited to, biliary tract cancer; brain cancer, including glioblastomas and medulloblastomas; breast cancer; cervical cancer; choriocarcinoma; colon cancer; endometrial cancer; esophageal cancer; gastric cancer; hematological neoplasms, including acute and chronic lymphocytic and myelogenous leukemia, multiple myeloma, AIDS associated leukemias and adult T-cell leukemia lymphoma; intraepithelial neoplasms, including Bowen's disease and Paget's disease; liver cancer; lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreatic cancer (see Jäger et. al., Mol Pharmacol 65:630-638, 2004); prostate cancer (see Parihar et. al., Eur. J. Pharmacol. 471 (2003) 157-164); rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma (teratomas, choriocarcinomas)), stromal tumors and germ cell tumors; thyroid cancer, including thyroid adenocarcinoma and medullar carcinoma; and renal cancer including adenocarcinoma and Wilms tumor.

The compounds of the invention are useful with hormone dependent and also with nonhormone dependent cancers. They also are useful with prostate and nonprostate cancers and with breast and nonbreast cancers. They further are useful with multidrug resistant strains of cancer.

In addition to the particular disorders enumerated above, the invention is also useful in treating or preventing dermatological diseases including keloids, hypertrophic scars, seborrheic dermatosis, papilloma virus infection (e.g., producing verruca vulgaris, verruca plantaris, verruca plan, condylomata, etc.), eczema and epithelial precancerous lesions such as actinic keratosis. Other T-cell mediated inflammatory disease states may also benefit from the methods described herein including arthritis, asthma and other respiratory ailments mediated by the inflammatory process; proliferative glomerulonephritis; lupus erythematosus (and other auto-immune diseases); scleroderma; temporal arthritis; thromboangiitis obliterans; mucocutaneous lymph node syndrome; and other pathologies mediated by growth factors including uterine leiomyomas.

These and other objects and advantages of the present invention will be apparent from the detailed description and examples that follow. All publications, patents and patent applications are incorporated herein by reference in their entirety.

### BRIEF DESCRIPTION OF THE DRAWINGS

FIG. 1 illustrates the in vitro conversion of a prodrug to an active IK1 blocker.

FIG. 2 illustrates the average concentration of a sulfone-containing phosphate prodrug and the corresponding active compound in the eyes of rabbits after topical administration of the sulfone-containing phosphate prodrug.

FIG. 3 illustrates the effects of a sulfone-containing phosphate prodrug on rabbit intraocular pressure: (A) ˜0.1% of a sulfone-containing phosphate prodrug in vehicle; (B) ˜0.5% of a sulfone-containing phosphate prodrug in vehicle; and (C) vehicle control.

### DETAILED DESCRIPTION OF THE INVENTION

Abbreviations and Definitions:

The abbreviations used herein have their conventional meaning within the chemical and biological arts. For example: DMAP, dimethylaminopyridine, DCC, dicyclohexylcarbodiimide, Et₃N, triethylamine; MeOH, methanol; and DMSO, dimethylsulfoxide; SAD-1, a transgenic mouse model of sickle cell disease as described by Trudel et al., EMBO J., 10(11): 3157-3165 (1991).

“Blocking” and “inhibiting,” are used interchangeably herein to refer to the partial or full blockade of an intermediate conductance, calcium activated potassium channel by one or more compound(s) of the invention.

Where substituent groups are specified by their conventional chemical formulae, written from left to right, they equally encompass the chemically identical substituents which would result from writing the structure from right to left, e.g., —CH₂O— is intended to also recite —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂,—S(O)—CH₃, —CH₂—CH₂—S(O)₂—CH₃, —CH═CH—O—CH₃, —Si(CH₃)₃, —CH₂—CH=N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent radical derivatives of “cycloalkyl” and “heterocycloalkyl,” respectively.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. The terms “arylene” and “heteroarylene” refer to the divalent radical derivatives of “aryl” and “heteroaryl,” respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—R′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q′}—U—, wherein T and U are independently —NR—, —O—, —CRR′— or a single bond, and q′ is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” is meant to include oxygen (O), nitrogen (N), sulfur (S) and silicon (Si).

A “substituent group,” as used herein, means a group selected from the following moieties:
(A) —OH, —NH₂, —SH, —CN, —CF₃, oxy, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxy, —OH, —NH₂, —SH, —CN, —CF₃, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxy, —OH, —NH₂, —SH, —CN, —CF₃, halogen, unsubstituted alkyl, unsubstituted heteroalkyl, unsubstituted cycloalkyl, unsubstituted heterocycloalkyl, unsubstituted aryl, unsubstituted heteroaryl, and
   (b) alkyl, heteroalkyl, cycloalkyl, heterocycloalkyl, aryl, or heteroaryl, substituted with at least one substituent selected from oxy, —OH, —NH₂, —SH, —CN, —CF₃, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2- to 20-membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The term “sickle cell disease” means a red blood cell disorder characterized by the presence of one or more mutated hemoglobin genes. Exemplary mutated hemoglobin genes include, for example, β^{6Glu→Val} (Hemoglobin S), β^{6Glu→Lys} (Hemoglobin C), β2^{6Glu→Val} (Hemoglobin E), β^{98Val→Met} (Hemoglobin Köln), β^{99Asp→His} (Hemoglobin Yakima), β^{102Asn→Lys} (Hemoglobin Kansas), or combinations thereof. Sickle cell diseases include, for example, sickle cell trait (the heterozygous state of hemoglobin S), sickle cell anemia (the homozygous state of hemoglobin S), hemoglobin SC disease (hemoglobin S present with hemoglobin C), hemoglobin SD disease (hemoglobin S present with hemoglobin D), S/β° thalassemia (hemoglobin S with a β° thalassemia mutation), and S/β⁺ thalassemia (hemoglobin S with a β⁺ thalassemia mutation). Current treatments for sickle cell diseases include, for example, administration of compounds such as antisickling agents (e.g. hydroxyurea), erythropoietin, and/or antibiotics (e.g. ceftriaxone and erythromycin), and allogenic bone marrow transplantation.

The term “glaucoma” refers to an optic neuropathy or degenerative state usually associated with elevation of intraocular pressure. See, Shields, TEXTBOOK OF GLAUCOMA (4^{th} Ed.), 1997, Lippincott, Williams and Wilkins, which is incorporated herein by reference. The mechanism by which elevated eye pressure injures the optic nerve is not well understood. It is known that axons entering the inferotemporal and superotemporal aspects of the optic disc are damaged. As fibers of the disc are destroyed, the neural rim of the optic disc shrinks and the physiologic cup within the optic disc enlarges. A term known as pathologic “cupping” refers to this shrinking and enlarging process. Although it is possible to measure the cup-to-disc ratio, it is not a useful diagnostic tool because it varies widely in the population. However, it can be used to measure the progression of the disease in an individual by a series of measurements in a time period.

Glaucoma is not a single disease but a group of conditions with various causes. Ultimately glaucoma can lead to optic nerve damage and the loss of visual function. It is not unusual for persons who exhibit gradual development of intraocular pressure to exhibit no symptoms until the end-stage of the disease is reached.

The term “open angle glaucoma”—refers to a chronic type of glaucoma. Occurring in approximately 1% of Americans, open-angle glaucoma is the most common type of glaucoma. Open-angle glaucoma is characterized by a very gradual, painless rise of pressure within the eye. Subjects with open-angle glaucoma exhibit no outward manifestations of disease until irreversible vision impairment.

“Normal tension glaucoma” commonly referred to as low tension glaucoma is a form of open angle glaucoma that accounts for about ⅓ of open-angle glaucoma cases in the United States.

“Angle closure glaucoma” is a glaucoma most prevalent in people who are far-sighted. In angle closure glaucoma, the anterior chamber of the eye is smaller than average hampering the ability of the aqueous humor to pass between the iris and the lens on its way to the anterior chamber, causing fluid pressure to build up between the iris.

“Acute glaucoma” is caused by a sudden increase in intraocular pressure. This intense rise in pressure is accompanied by severe pain. In acute glaucoma, there are outward manifestations of the disease including red eye, cornea swelling and clouding over.

The term “pigmentary glaucoma” refers to a hereditary condition which develops more frequently in men than in woman and begins in the twenties or thirties. pigmentary glaucoma affects persons of near-sightedness. Myopic eyes have a concave-shaped iris creating an unusually wide angle. The wideness of the angle causes the pigment layer of the eye to rub on the lens when the pupil constricts and dilates during normal focusing. The rubbing action ruptures the cells of the iris pigment epithelium, thereby releasing pigment particles into the aqueous humor and trabecular meshwork. If pigment plugs the pores of the trabecular meshwork, drainage is inhibited.

The term “exfoliation syndrome” refers to a type of glaucoma most common in persons of European descent. Exfoliation syndrome is characterized by a whitish material that builds on the lens of the eye. Movement of the iris causes this material to be rubbed off the lens along with some pigment from the iris. Both the pigment and the whitish exfoliation material clog the meshwork, inhibiting drainage of the aqueous humor.

The term “trauma related glaucoma” refers to a type of glaucoma caused by an external force acting upon the eye, i.e., chemical burn, blow to the eye. Trauma related glaucoma occurs when this external force causes a mechanical disruption or physical change with in the eye's drainage system.

“Congenital glaucoma” occurs in about 1 in 10,000 births. It may appear up until age 4. Primary congenital glaucoma is due to abnormal development of the trabecular meshwork. Congenital glaucoma can be hereditary as well as non-hereditary. In congenital glaucoma, the eye enlarges or the cornea becomes hazy. The stretching of the cornea causes breaks to occur in the inner lining. The breaks allow aqueous humor to enter the cornea causing it to swell. As the cornea continues to stretch, more aqueous humor is let in and there is an increase in edema and haze in the cornea.

The term “Sturge-Weber Syndrome” refers to a rare syndrome characterized by a facial birthmark port wine in color. The birthmark is associated with an abnormal blood vessels on the surface of the brain. These vascular malformations may affect the eyelids, sclera, conjunctiva, and iris. One third of patients with Sturge-Weber syndrome suffer from increased intraocular pressure. This increased pressure leads to glaucoma. A vascular malformation of the sclera causes elevated pressure in the veins. This elevated pressure in the veins drains the eye thereby causing the intraocular pressure to rise and resulting in damage to the drainage system of the eye.

The term “uveitis” refers to a disease characterized by inflammation of the choroid, ciliary body and iris. In anterior uveitis, a decrease in aqueous humor formation may cause dangerously low levels of pressure within the eye. In other forms of uveitis, i.e., posterior uveitis, the intraocular pressure is elevated. The elevation may be caused by active inflammation, insufficient antiinflammatory therapy, excessive corticosteroid use or insufficient glaucoma therapy. If the inflammation is chronic and not properly controlled, it can lead to trabecular cell death.

The term “chronic elevation” refers to increased pressure caused by a condition that is reoccurring and not treatable.

The term “acute elevation” refers to a sudden increase in intraocular eye pressure. The sudden rise can occur within hours and causes pain within the eye and may even cause nausea and vomiting

The term “gradual elevation” refers to a slow increase of pressure within the eye. There are no symptoms associated with the increased rise.

An “ophthalmically acceptable carrier” is a carrier that has substantially no long term or permanent detrimental effect on the eye to which it is administered.

A “fused ring” is a cycloalkyl, heterocycloalkyl, aryl, or heteroaryl ring the shares at least 2 vertices with a cycloalkyl, heterocycloalkyl, aryl, or heteroaryl ring parent compound.

The term “prodrug,” as used herein, represents compounds which are rapidly transformed in vivo to parent compounds having formula (I), for example, by hydrolysis in blood. A thorough discussion is provided in T. Higuchi and V. Stella, Prodrugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are hereby incorporated by reference.

The term “pharmaceutically acceptable prodrugs” as used herein represents those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention.

Where a single substituent appears more than once in a compound and can be selected from a group of possible chemical moieties, each occurrence of the substituent is optionally different. Thus, a single substituent may be referred to as being “independently selected from” a group of substituents. While the term “independently selected from” refers to the possibility of each occurrence of a substituent being optionally different, the absence of this phrase does not necessarily mean that each occurrence of a single substituent is not optionally different.

Introduction

It has been discovered that, surprisingly, sulfone-containing compounds having a prodrug modification group effectively block IK1 channels after undergoing a chemical alteration of the prodrug moiety. The sulfone-containing prodrugs of the present invention include a prodrug modification group, which is capable of being chemically altered (e.g. partially or completely cleaved) by an enzyme thereby forming an active IK1 potassium channel blocker.

As discussed above, the blockade of the intermediate conductance calcium activated potassium channel is a powerful therapeutic approach for the treatment of disease states in which said channel plays a therapeutically relevant role as a drug target. Representative diseases that may be treated by inhibition of the intermediate conductance, calcium activated potassium channel include, but are not limited to sickle cell disease and glaucoma.

The present invention is illustrated by reference to the use of the compounds of the invention in treating sickle cell disease and glaucoma. The focus on the two selected diseases is for clarity of illustration only and is not intended to define or otherwise limit the scope of the present invention.

The prevention of sickle cell dehydration via inhibition of the intermediate conductance, calcium activated potassium channel (i.e., the Gardos channel) is useful in the treatment and/or prevention of sickle cell disease. Moreover, physiological studies show that intermediate conductance calcium activated potassium channels play a role in secretion of Cl⁻ and water from epithelial tissue. Given that the intraocular pressure of the eye is maintained, in part, by secretion of aqueous humor, the inhibition of aqueous humor secretion by an antagonist of the intermediate conductance, calcium activated potassium channel reduces intraocular pressure. For example, in rabbits, topical application of a compound of the invention was demonstrated to result in a dose-dependent, long duration reduction in intraocular pressure. Thus, the blockade of intermediate conductance, calcium activated potassium channels in the eye is of benefit for the treatment of glaucoma.

The present invention provides sulfone compounds, compositions containing these compounds, and methods for using these compounds and compositions to decrease ion flux in intermediate conductance, calcium activated potassium channels. Inhibition of said channel reduces mammalian cell proliferation, intraocular pressure, erythrocyte dehydration, sickle cell dehydration, and delays the occurrence of acute sickle cell episodes. Thus, the present invention also provides methods of using the compounds of the invention to treat and prevent diseases in which inhibition of ion flux through intermediate conductance, calcium activated potassium channels may prove beneficial.

### DESCRIPTION OF THE EMBODIMENTS

I. Prodrug Modulators of Intermediate Conductance Calcium Activated Potassium Channels

In one aspect, the present invention provides prodrug compounds according to Formula (I):

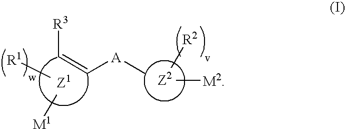

In Formula (I), ring system Z¹ is selected from aryl, and 5-membered heterocycloalkyl. Ring system Z² is selected from aryl, heteroaryl, (C₅-C₇)cycloalkyl and 5 to 7 membered heterocycloalkyl.

The symbols w and v are integers independently selected from 0 to 4. One of skill in the art will immediately recognize that the number of R¹ substituents and R² substituents may not exceed the number of available attachment points on ring systems Z¹ and Z² respectively, according to the normal rules of chemical valency. For example, where Z¹ is phenyl, w is an integer selected from 0, 1, 2, and 3; and where Z² is phenyl, v is an integer selected from 0, 1, 2, 3, and 4. In some embodiments, w is 1 or 2. The symbol v may be 1 or 2. In other embodiments, w and v are 1. The symbols w and v may also be 0. In some embodiments, w is 0 and v is 1.

The symbols R¹ and R² represent moieties independently selected from —NH₂, —NO₂, —CF₃, —OCF₃, hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a fused ring, and OR⁹. R⁹ is selected from hydrogen, —CF₃, substituted or unsubstituted alkyl and substituted or unsubstituted heteroalkyl.

R³ is selected from —C(O)OR^{3A}, 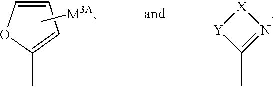
R^{3A} is selected from methyl, ethyl and —CF₃. X is selected from —N═N—, —N═C(R^{3B})—, —C(R^{3B}R^{3C})—C(R^{3D}R^{3E}) and —C(R^{3B})═C(R^{3C})—. R^{3B}, R^{3C}, R^{3D}, and R^{3E} are independently selected from hydrogen, substituted and unsubstituted lower alkyl, -M^{3B} and —CF₃. In some embodiments, only one of R^{3B}, R^{3C}, R^{3D}, or R^{3E} may be M^{3B} simultaneously. Y is selected from —O—, —N(M^{3C})— and —S—.

A is selected from -L¹-N(M⁴)-S(O)₂-L²-, and -L-S(O)_{q}-L²-. The symbol q is an integer selected from 0 to 2. L¹ and L² are independently selected from a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene.

M¹, M², M^{3A}, and M^{3B} are independently selected from hydrogen and -L⁵-X⁵—R⁵. L⁵ is independently selected from a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene. X⁵ is independently selected from —O— and —N(R⁶)—. R⁶ is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R⁵ is independently selected from —C(O)—R^{5A} and 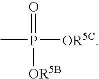
R^{5B} and R^{5C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R^{5A} is selected from -L^{5A1}-NR^{5A1}R^{5A2} and —OR^{5A3}. L^{5A1} is selected from a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene. R^{5A1} and R^{5A3} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{5A2} is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, and —C(O)R^{5A5}. R^{5A5} is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

M^{3C} and M⁴ are independently selected from hydrogen, substituted or unsubstituted lower alkyl, —CF₃, —C(O)—R^{7A} and 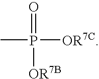
In some embodiments, M^{3C} and M⁴ are independently selected from hydrogen, —C(O)—R^{7A} and 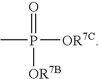
R^{7B} and R^{7C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R^{7A} is a selected from -L^{7A1}-NR^{7A1}R^{7A2}, and —OR^{7A3}. L^{7A1} is a selected from a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene. R^{7A1} and R^{7A3} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{7A2} is independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, and —C(O)R^{7A5}. R^{7A5} is a member selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In some embodiments, at least one of M¹, M², M^{3A}, or M^{3B} is -L⁵-X⁵—R⁵, or at least one of M^{3C} or M⁴ is —C(O)—R^{7A} or 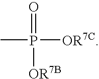
In other embodiments, only one of M¹, M², M^{3A}, M^{3B}, M^{3C} or M⁴ may be -L⁵-X⁵—R , or —C(O)—R^{7A} or 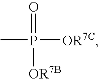
respectively.

In an exemplary embodiment, A is selected from —N(M⁴)-S(O)₂—, -L¹-S(O)_{q}—, and —S(O)_{q}-L²-. L¹ and L² may be a bond. L¹ and L² may also be —C(R^{8A}R^{8B})—, where R^{8A} and R^{8B} are independently selected from hydrogen, substituted and unsubstituted lower alkyl, —OR^{4C} and —CF₃. R^{4C} is a member selected from hydrogen, and substituted or unsubstituted lower alkyl.

In some embodiment of the present invention, A is —N(M⁴)-S(O)₂—. In other embodiments, A is —N(H)—S(O)₂—.

R³ may represent

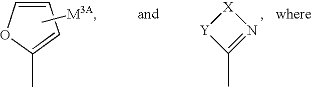

X is selected from —N′C(R^{3B})—, —C(R^{3B}R^{3C})—C(R^{3D}R^{3E})— and —C(R^{3B})═C(R^{3C})—, and Y is selected from O and S.

Z¹ may be selected from phenyl and thiophenyl. Z² may be selected from 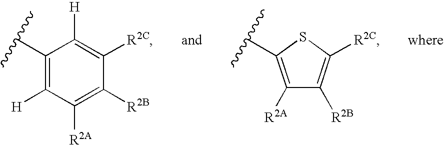
where R^{2A}, R^{2B} and R^{2C} are independently selected from hydrogen, halogen, lower alkyl, —OR^{2D}, —CF₃, NO₂, Cl, and M². In some embodiments, only one of R^{2A}, R^{2B} and R^{2C} may be M² simultaneously. In some embodiments, at least one of R^{2A}, R^{2B} and R^{2C} is halogen, lower alkyl, —OR^{2D}, —CF₃, NO₂, Cl, or M². In some embodiments, R^{2A}, R^{2B} and R^{2C} are independently selected from halogen, lower alkyl, —OR^{2D}, —CF₃, NO₂, Cl, and M². Here, R^{2D} is a member selected from the group consisting of H, substituted or unsubstituted lower alkyl, and —CF₃.

In a related embodiment, M² is -L⁵-X⁵—R⁵, where L⁵ is a bond and X⁵ is —O—. Z² may also be

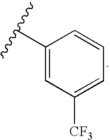

R¹ and R² may be selected from —NO₂, —CF₃, hydrogen, halogen, substituted or unsubstituted (C₁-C₁₀)alkyl, substituted or unsubstituted 2 to 10 membered heteroalkyl, substituted or unsubstituted (C₅-C₇) cycloalkyl, substituted or unsubstituted 5 to 7 membered heterocycloalkyl, substituted or unsubstituted aryl, substituted or unsubstituted heteroaryl, a fused ring heterocycloalkyl, and OR⁹. In an exemplary embodiment, R¹ and R² are independently selected from —NO₂, —CF₃, hydrogen, halogen, unsubstituted (C₁-C₁₀)alkyl, unsubstituted 2 to 10 membered heteroalkyl, unsubstituted (C₅-C₇) cycloalkyl, unsubstituted 5 to 7 membered heterocycloalkyl, unsubstituted aryl, unsubstituted heteroaryl, OR⁹, and C(O)OR¹⁰. In a related embodiment, R¹ and R² are independently selected from —NO₂, —CF₃, hydrogen, halogen, unsubstituted (C₁-C₁₀)alkyl, unsubstituted 2 to 10 membered heteroalkyl, OR⁹, and C(O)OR¹⁰. In another related embodiment, R¹ and R² are independently selected from —NO₂, —CF₃, F, Cl, hydrogen, methyl, ethyl, —C(O)OCH₃, —OCH₃, and —OCF₃.

R⁹ may be selected from —CF₃, substituted or unsubstituted (C₁-C₅)alkyl and substituted or unsubstituted 2 to 5 membered heteroalkyl. In some embodiments, R⁹ is selected from —CF₃, unsubstituted (C₁-C₅)alkyl and unsubstituted 2 to 5 membered heteroalkyl. In a related embodiment, R⁹ is selected from —CF₃, unsubstituted (C₁-C₅)alkyl and unsubstituted 2 to 5 membered heteroalkyl.

R¹⁰ may be an unsubstituted (C₁-C₁₀)alkyl. In some embodiments, R¹⁰ is an unsubstituted (C₁-C₅)alkyl.

Prodrug Modification Groups

A variety of prodrug modification groups are useful in the present invention. Prodrug modification groups useful in the present invention include -L⁵-X⁵—R⁵, —C(O)—R^{7A} and 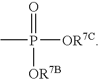
In some embodiments, the compound of Formula (I) includes at least one of these prodrug modification groups. In other embodiments, the compound of Formula (I) includes only one of these prodrug modification groups.

The linker group L⁵ may be selected from a bond, substituted or unsubstituted (C₁-C₁₀) alkylene, and substituted or unsubstituted 2 to 10 membered heteroalkylene. In some embodiments, L⁵ is independently selected from a bond, unsubstituted (C₁-C₁₀) alkylene, and unsubstituted 2 to 10 membered heteroalkylene. Alternatively, L⁵ is simply a bond.

X⁵ may be independently selected from —O— and —N(R⁶)— where R⁶ is selected from hydrogen, substituted or unsubstituted (C₁-C₁₀) alkyl, and substituted or unsubstituted 2 to 10 membered heteroalkyl. In an exemplary embodiment, R⁶ is selected from unsubstituted (C₁-C₁₀) alkyl and unsubstituted 2 to 10 membered heteroalkyl. In other embodiments, X⁵ is —O—.

R⁵ moieties may be selected from —C(O)—R^{5A} and 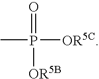
R^{5B} and R^{5C} groups may be selected from hydrogen, substituted or unsubstituted (C₁-C₁₀) alkyl, and substituted or unsubstituted 2 to 10 membered heteroalkyl. In some embodiments, R^{5B} and R^{5C} groups are selected from hydrogen, unsubstituted (C₁-C₁₀) alkyl, and unsubstituted 2 to 10 membered heteroalkyl. In a related embodiments, R^{5B} and R^{5C} groups are elected from hydrogen, unsubstituted (C₁-C₅) alkyl, and unsubstituted 2 to 5 membered heteroalkyl. Alternatively, R^{5B} and R^{5C} are both hydrogen.

R^{5A} may be selected from -L^{5A1}NR^{5A1}R^{5A2} and OR^{5A3}. In some embodiments, R^{5A} is -L^{7A1}-NR^{7A1}R^{7A2}. The linker L^{5A1} may be selected from substituted or unsubstituted (C₁-C₁₀) alkylene, and substituted or unsubstituted 2 to 10 membered heteroalkylene. In an exemplary embodiment, L^{5A1} is selected from unsubstituted (C₁-C₁₀) alkylene, and unsubstituted 2 to 10 membered heteroalkylene. In a related embodiment, L^{5A1} is selected from unsubstituted (C₁-C₅) alkylene, and unsubstituted 2 to 5 membered heteroalkylene. In other related embodiments, L^{5A1} is a bond.

R^{5A1} and R^{5A3} may be independently selected from hydrogen, substituted or unsubstituted (C₁-C₁₀) alkyl, and substituted or unsubstituted 2 to 10 membered heteroalkyl. In some embodiments, R^{5A1} and R^{5A3} are independently selected from hydrogen, unsubstituted (C₁-C₁₀) alkyl, and unsubstituted 2 to 10 membered heteroalkyl. In a related embodiment, R^{5A1} and R^{5A3} are independently selected from hydrogen, unsubstituted (C₁-C₅) alkyl, and unsubstituted 2 to 5 membered heteroalkyl. In another related embodiment, R^{5A1} and R^{5A3} are independently selected from hydrogen and unsubstituted (C₁-C₅) alkyl.

R^{5A2} may be selected from hydrogen, substituted or unsubstituted (C₁-C₁₀) alkyl, and substituted or unsubstituted 2 to 10 membered heteroalkyl, and —C(O)R^{5A5}. In an exemplary embodiment, R^{5A2} is selected from hydrogen, unsubstituted (C₁-C₅) alkyl, unsubstituted 2 to 10 membered heteroalkyl, and —C(O)R^{5A5}. In a related embodiment, R^{5A2} is selected from hydrogen, unsubstituted (C₁-C₅) alkyl.

Useful R^{5A5} groups include those selected from hydrogen, substituted or unsubstituted (C₁-C₁₀) alkyl, and substituted or unsubstituted 2 to 10 membered heteroalkyl. R^{5A5} may also be selected from hydrogen, unsubstituted (C₁-C₁₀) alkyl, and unsubstituted 2 to 10 membered heteroalkyl. In an exemplary embodiment, R^{5A5} is selected from hydrogen, unsubstituted (C₁-C₅) alkyl, and unsubstituted 2 to 5 membered heteroalkyl.

M^{3C} and M⁴ may be independently selected from hydrogen, —C(O)—R^{7A} and 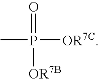
In some embodiments, M^{3C} and M⁴ are independently selected form hydrogen and —C(O)—R^{7A}.

R^{7B} and R^{7C} groups may be selected from hydrogen, substituted or unsubstituted (C₁-C₁₀) alkyl, and substituted or unsubstituted 2 to 10 membered heteroalkyl. In some embodiments, R^{7B} and R^{7C} groups are selected from hydrogen, unsubstituted (C₁-C₁₀) alkyl, and unsubstituted 2 to 10 membered heteroalkyl. In a related embodiments, R^{7B} and R^{7C} groups are elected from hydrogen, unsubstituted (C₁-C₅) alkyl, and unsubstituted 2 to 5 membered heteroalkyl. Alternatively, R^{7B} and R^{7C} are both hydrogen.

R^{7A} may be selected from -L^{7A1}-NR^{7A1}R^{7A2} and —OR^{7A3}. In some embodiments, R^{7A} is -L^{7A1}-NR^{7A1}R^{7A2}. The linker L^{7A1} may be selected from substituted or unsubstituted (C₁-C₁₀) alkylene, and substituted or unsubstituted 2 to 10 membered heteroalkylene. In an exemplary embodiment, L^{7A1} is selected from unsubstituted (C₁-C₁₀) alkylene, and unsubstituted 2 to 10 membered heteroalkylene. In a related embodiment, L^{7A1} is selected from unsubstituted (C₁-C₅) alkylene, and unsubstituted 2 to 5 membered heteroalkylene. In other related embodiments, L^{7A1} is unsubstituted (C₁-C₅) alkylene.

R^{7A1} and R^{7A3} may be independently selected from hydrogen, substituted or unsubstituted (C₁-C₁₀) alkyl, and substituted or unsubstituted 2 to 10 membered heteroalkyl. In some embodiments, R^{7A1} and R^{7A3} are independently selected from hydrogen, unsubstituted (C₁-C₁₀) alkyl, and unsubstituted 2 to 10 membered heteroalkyl. In a related embodiment, R^{7A1} and R^{7A3} are independently selected from hydrogen, unsubstituted (C₁-C₅) alkyl, and unsubstituted 2 to 5 membered heteroalkyl. In another related embodiment, R^{7A1} and R^{7A3} are independently selected from hydrogen and unsubstituted (C₁-C₅) alkyl.

R^{7A2} may be selected from hydrogen, substituted or unsubstituted (C₁-C₁₀) alkyl, and substituted or unsubstituted 2 to 10 membered heteroalkyl, and —C(O)R^{7A5}. In an exemplary embodiment, R^{7A2} is selected from hydrogen, unsubstituted (C₁-C₅) alkyl, unsubstituted 2 to 10 membered heteroalkyl, and —C(O)R^{7A5}. In a related embodiment, R^{7A2} is selected from hydrogen, unsubstituted (C₁-C₅) alkyl.

R^{7A5} groups may include those selected from hydrogen, substituted or unsubstituted (C₁-C₁₀) alkyl, and substituted or unsubstituted 2 to 10 membered heteroalkyl. R^{7A5} may also be selected from hydrogen, unsubstituted (C₁-C₁₀) alkyl, and unsubstituted 2 to 10 membered heteroalkyl. In an exemplary embodiment, R^{7A5} is selected from hydrogen, unsubstituted (C₁-C₅) alkyl, and unsubstituted 2 to 5 membered heteroalkyl.

In some embodiments, M^{3C} and M⁴ may be independently selected from hydrogen, —C(O)—R^{7A} and 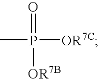
and M¹, M², M^{3A}, and M^{3B} are independently selected from hydrogen and -L⁵-X⁵—R⁵. In a related embodiment, only one of M¹, M², M^{3A}, M^{3B}, M^{3C} and M⁴ is not hydrogen. Thus, in some embodiments, only one of M¹, M², M^{3A}, M^{3B}, M^{3C} and M⁴ is a prodrug modification group.

In some embodiments, M², M^{3A}, M^{3B}, M^{3C}, and M⁴ are hydrogen and M¹ is -L⁵-X⁵—R⁵. R⁵ may be selected from —C(O)—R^{5A} and 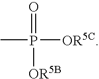
R^{5B} and R^{5C} may be hydrogen. R^{5A} may be -L^{5A1}-NR^{5A1}R^{5A2}. L^{5A1} may be unsubstituted (C₁-C₅) alkylene. R^{5A1} and R^{5A2} may be members independently selected from hydrogen, and unsubstituted (C₁-C₅) alkyl.

In other embodiments, M², M^{3A}, M^{3B}, M^{3C}, and M⁴ are hydrogen and M¹ is -L⁵-X⁵—R⁵, where L⁵ is a bond, X⁵is —O—, and R⁵ is 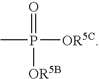
R^{5B} and R^{5C} may both be hydrogen.

In other embodiments, M², M^{3A}, M^{3B}, M^{3C}, and M⁴ are hydrogen and M¹ is -L⁵-X⁵—R⁵, where L⁵ is a bond, X⁵ is —O—, and R⁵ is —C(O)—R^{5A}. R^{5A} may be -L^{5A1}-NR^{5A1}R^{5A2}. L^{5A1} may be C₁—, C₂—, C₃—, C₄—, or C₅-unsubstituted alkylene. R^{5A1} and R^{5A2} may be selected from hydrogen, methyl, ethyl, propyl, butyl, and pentyl.

In other embodiments, M², M^{3A}, M^{3B}, M^{3C}, and M¹ are hydrogen and M⁴ is —C(O)—R^{7A}. R^{7A} may be -L^{7A1}-NR^{7A1}R^{7A2}. L^{7A1} may be unsubstituted (C₁-C₅) alkylene. R^{7A1} and R^{7A2} may be members independently selected from hydrogen, unsubstituted (C₁-C₅) alkyl.

In some embodiments, each substituted moiety described above for the compounds of the present invention (e.g. the compound of Formula (I), including the prodrug modification groups discussed above) is substituted with at least one substituent group. The term “substituent group,” as used herein, is defined in detail above in the “Abbreviations and Definitions” section. More specifically, in some embodiments, each substituted alkylene, substituted heteroalkylene, substituted cycloalkylene, substituted heterocycloalkylene, substituted arylene, substituted heteroarylene, substituted lower alkyl, substituted alkyl, substituted heteroalkyl, substituted cycloalkyl, substituted heterocycloalkyl, substituted aryl, substituted heteroaryl, substituted alkylene, and/or substituted heteroalkylene, described above are substituted with at least one substituent group. Each substituent group is optionally different. In other embodiments, at least one or all of these groups are substituted with at least one size-limited substituent group. Alternatively, at least one or all of these groups are substituted with at least one lower substituent group. Size-limited substituent groups and lower substituent groups are both defined in detail above in the “Abbreviations and Definitions” section.

In other embodiments, each substituted or unsubstituted alkyl is a substituted or unsubstituted C₁-C₂₀ alkyl, and each substituted or unsubstituted heteroalkyl is a substituted or unsubstituted 2- to 20-membered heteroalkyl.

Alternatively, each substituted or unsubstituted alkyl is a substituted or unsubstituted C₁-C₈ alkyl, and each substituted or unsubstituted heteroalkyl is a substituted or unsubstituted 2- to 8-membered heteroalkyl.

In an exemplary embodiment, each substituted alkyl, substituted heteroalkyl, substituted cycloalkyl, substituted heterocycloalkyl, substituted aryl, substituted fused ring, and substituted heteroaryl are substituted only with at least one substituent independently selected from —OH, —NH₂, —SH, —SO₂, —NO₂, —CF₃, —OCF₃, —CN, halogen, unsubstituted (C₁-C₃₀)alkyl, unsubstituted 2 to 30 membered heteroalkyl (including branched heteroalkyls), unsubstituted (C₅-C₇) membered cycloalkyl, unsubstituted 5 to 7 membered heterocycloalkyl, unsubstituted aryl, unsubstituted heteroaryl. In addition, each substituted alkylene, substituted heteroalkylene, substituted cycloalkylene, substituted heterocycloalkylene, substituted arylene, and substituted heteroarylene are substituted only with at least one substituent independently selected from —OH, —NH₂, —SH, —SO₂, —NO₂, —CF₃, —OCF₃, —CN, halogen, unsubstituted (C₁-C₃₀)alkyl, unsubstituted 2 to 30 membered heteroalkyl (including branched heteroalkyls), unsubstituted (C₅-C₇) membered cycloalkyl, unsubstituted 5 to 7 membered heterocycloalkyl, unsubstituted aryl, unsubstituted heteroaryl.

Preparation of Intermediate Conductance Calcium Activated Potassium Channel Blockers

Compounds of the present invention can be prepared using readily available starting materials or known intermediates. For example, furan derivatized bis-aryl sulfonamides are readily prepared the method of Scheme A:

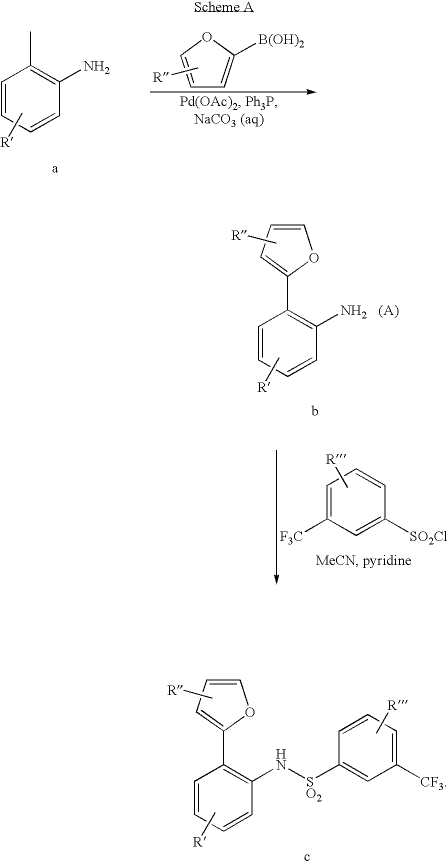

In Scheme A, and each of the succeeding schemes, each of the reaction components can bear one or more substituents (“R groups”) other than a locus of reaction. The symbols R′, R″, R′″, etc. generally represent substituents for aryl or heteroaryl groups as described in the definitions section herein, or may correspond to the substituents described above in Section I (e.g. R′ may correspond to R′ and R″ may correspond to R²).

In scheme A, the iodo aniline substrate a is coupled with the furan moiety via a Pd mediated reaction with a boronic acid derivative to afford compound b. The resulting adduct is reacted with an activated sulfonic acid derivative to produce adduct c.

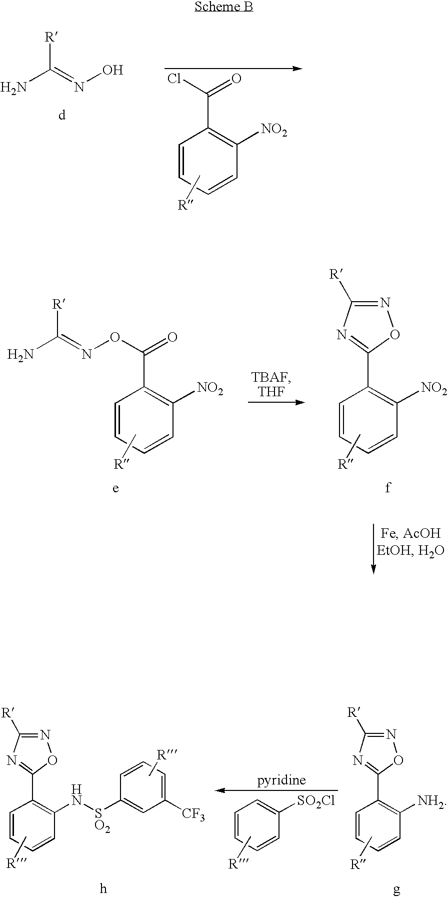

Scheme B sets out an exemplary route to oxadiazolyl-containing compounds of the invention. Thus, amidine d is acylated with a benzoyl chloride species, affording compound e. Compound e is cyclized to compound f. The nitro group of compound f is reduced and the resulting amine is converted to the correspond sulfonamide h.

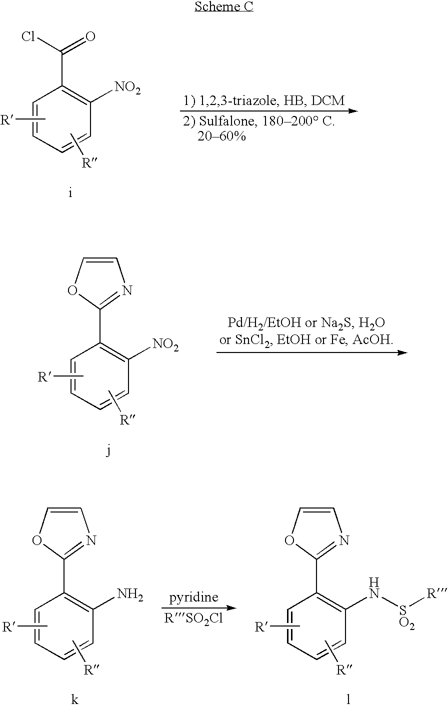

Scheme C sets forth a representative route to oxazole-containing compounds of the invention. Acyl halide i is converted to oxazole j by the action of triazole and sulfalone. The nitro group of j is reduced, affording the corresponding amine k, which is converted to a sulfonamide l by the action of an activated sulfonic acid derivative.

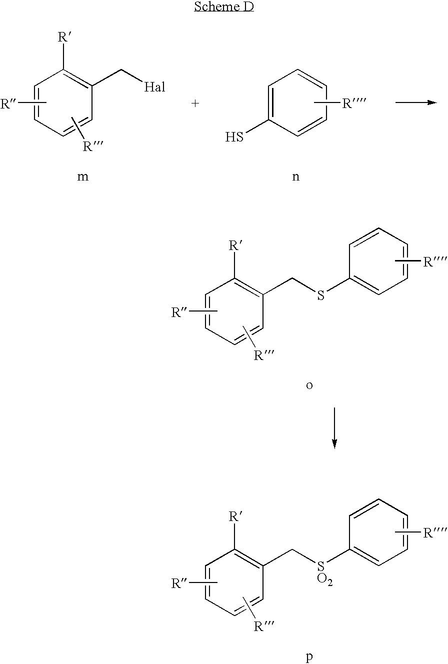

Scheme D provides an exemplary route to bis-aryl sulfonamides of the invention. Benzyl halide m is reacted with an appropriate thiol n, forming sulfide o, which is subsequently oxidized to sulfonamide p.

Methods for preparing dimers, trimers and higher homologs of small organic molecules, such as those of the present invention, as well as methods of functionalizing a polyfunctional framework molecule are well known to those of skill in the art. For example, an aromatic amine of the invention is converted to the corresponding isothiocyanate by the action of thiophosgene. The resulting isothiocyanate is coupled to an amine of the invention, thereby forming either a homo- or heterodimeric species. Alternatively, the isothiocyanate is coupled with an amine-containing backbone, such as polylysine, thereby forming a conjugate between a polyvalent framework and a compound of the invention. If it is desired to prepare a hetereofuntionalized polyvalent species, the polylysine is underlabeled with the first isothiocyanate and subsequently labeled with one or more different isothiocyanates. Alternatively, a mixture of isothiocyanates is added to the backbone. Purification proceeds by, for example, size exclusion chromatography, dialysis, nanofiltration and the like.

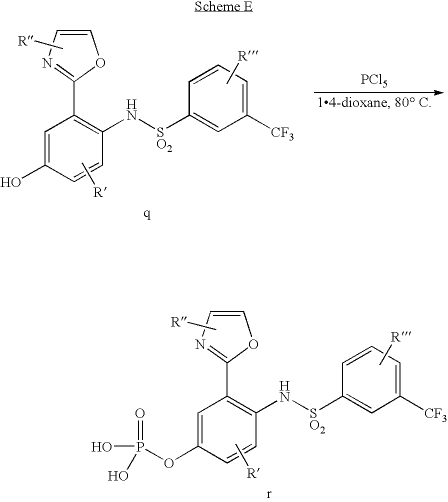

Scheme E illustrates a method for adding a phosphate prodrug modification to an oxazole containing sulfonamide. The oxazole-containing compound q is allowed to react with PCl₅ in anhydrous 1,4-dioxane followed by quenching with sat. NaHCO₃ to yield the phosphotrylated prodrug r.

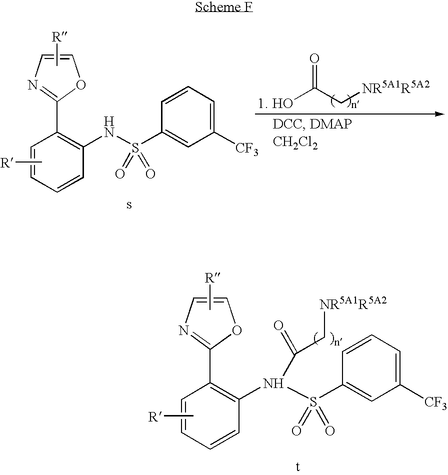

Scheme F illustrates a method for adding a carbonylamine prodrug modification to an oxazole-containing sulfonamide. Sulfonamide s is reacted with an appropriate carboxylic acid n, in the presence of DCC and DMAP to yield the carbonylamine prodrug t. In Scheme F, R^{5A1} and R^{5A2} are as defined above and n′ is an integer, typically from 1 to 20.

Compound Stability

The prodrug compounds of the present invention are useful as intermediate conductance, calcium activated potassium channel inhibitors, after undergoing, for example, enzymatic hydrolysis or chemical hydrolysis under physiological conditions thereby forming an active IK1 channel blocker. Typically, the prodrug modification group is wholly or partially cleaved by a degradative enzyme. A variety of degradative enzymes may chemically alter the prodrug modification group to form an active IK1 channel blocker. Exemplary enzymes include but are no limited to proteases, peptidases, esterases, amidases, glucuronidases, sulfatases, phosphatases, hydrolases, and others. In an exemplary embodiment, the enzyme alters the prodrug modification group in vivo.

IK1 channel blockers preferably exhibit both acceptable bioavailability and stability in vivo. The stability of the IK1 channel blockers in various biological milieus can be assayed by methods known in the art. In one embodiment, the stability of the compounds is assayed in an in vitro preparation. In a preferred embodiment, the in vitro preparation is a liver microsome preparation. The results of such in vitro assays provide data relevant to the in vivo stability of the compounds of the invention. Other in vitro assays useful in assaying the stability of the compounds of the invention are known in the art.

In addition to in vitro methods, in vivo methods such as pharmacokinetic studies can be performed in a range of animal models. One or more compounds of the invention can be administered to an animal, preferably a rat, at different dosages and/or by different routes (e.g., i.v., i.p., p.o). Blood, urine and/or feces samples can be collected at serial time points and the samples assayed for the presence and/or concentration of the compound(s) of the invention. The appearance of the corresponding IK1 channel blocker formed after chemical alteration of the prodrug moiety may also be measured (see examples section).

Any appropriate quantity can be utilized to compare data from different compounds. Exemplary quantities include, half-life, bioavailability, amount of prodrug and or IK1 channel blocker compound remaining intact after a predetermined time period and the like. In a preferred embodiment, the amount of IK1 channel blocker and prodrug remaining intact after a predetermined time period is utilized.

Any technique that allows the detection and, preferably, the quantitation of the active IK1 channel blocker compound(s) and/or the prodrug is appropriate for use in assaying the compounds of the invention. These methods include, but are not limited to, spectrometric methods (e.g., NMR (e.g., ¹⁹F NMR), MS, IR, UV/vis), chromatographic methods (e.g., LC, GC, HPLC) and hybrid methods utilizing both spectrometric and chromatographic methods (e.g., GC/MS, LC/MS, LC/MS/MS). Further, the methods can utilize detectable labels such as compounds of the invention that are labeled with radioisotopes (e.g., ³H, ¹⁵N, ¹⁴C) or fluorescent labels (e.g., fluorescein, rhodamine). Other methods for assaying the in vivo persistence of small organic molecules, particularly those applicable to bioactive molecules, will be apparent to those of skill in the art.

Compound Activity

To develop pharmaceutically useful intermediate conductance, calcium activated potassium channel inhibitors, candidate prodrug compounds must be capable of being hyrdrolyzed as described above to active IK1 channel blocker compounds that demonstrate acceptable activity towards the target channel. The activity of the IK1 channel blocker compounds towards these ion channels, such as the Gardos channel, can be assayed utilizing methods known in the art.

Compounds that decrease ion flow through intermediate conductance, calcium activated potassium channels are tested using biologically active channels, either recombinant or naturally occurring. Intermediate conductance, calcium activated potassium channels, preferably human channels, can be found in native cells, isolated in vitro, co-expressed or expressed in a cell, or expressed in membrane derived from a cell. Modulation by a compound of the invention is tested using standard in vitro or in vivo assays such as those well known in the art or as otherwise described herein. Compounds that decrease the flux of ions will cause a detectable decrease in the ion current density by decreasing the probability of the channel being open, by increasing the probability of it being closed, by decreasing conductance through the channel, and by hampering the passage of ions.

Decreased flux of potassium may be assessed by determining changes in polarization (i.e., electrical potential) of a cell which expresses, for example, the intermediate conductance, calcium activated potassium channel known as the Gardos channel. One method of determining changes in cellular polarization is the voltage-clamp technique e.g., the “cell attached” mode, the “inside out” mode, and the “whole cell” mode (see, e.g., Ackerman et al., New Engl. J. Med. 336:1575-1595 (1997)). Other known assays include radiolabeled rubidium flux assays and fluorescence assays using voltage-sensitive dyes. See, e.g., Vestergarrd-Bogind et al., J. Membrane Biol., 88:67-75 (1988); Danel et al., J. Pharmacol. Meth., 25:185-193 (1991); Holevinsky et al., J. Membrane Biology, 137:59-70 (1994). Assays for compounds capable of inhibiting or increasing potassium flux through the intermediate conductance, calcium activated potassium channel protein can be performed by application of the compounds to a bath solution in contact with and comprising cells having said channel. See, e.g., Blatz et al., Nature, 323:718-720 (1986); Park, J. Physiol., 481:555-570 (1994). Generally the compounds to be tested are present in the range from 1 pM to 100 mM. Changes in function of the channels can be measured in the electrical currents or ionic flux, or by the consequences of changes in currents and flux.

The effects of prodrugs forming the test IK1 channel blocker compounds upon the function of the channels can be measured by changes in the electrical currents or ionic flux or by the consequences of changes in currents and flux. Changes in electrical current or ion flux are measured either by increases or decreases in flux of cations such as potassium or rubidium ions. The cations can be measured in a variety of standard ways. They can be measured directly by concentration changes of the ions or indirectly by membrane potential or by radiolabeling of the ions. Consequences of the test compound on ion flux can be quite varied. Accordingly, any suitable physiological parameter can be used to assess the influence of a test compound on the channels of this invention. Changes in channel function can be measured by ligand displacement such as CTX release. When the functional consequences are determined using intact cells or animals, one can also measure a variety of effects such as transmitter release (e.g., dopamine), hormone release (e.g., insulin), transcriptional changes to both known and uncharacterized genetic markers (e.g., northern blots), cell volume changes (e.g., in red blood cells), immune-responses (e.g., T cell activation), changes in cell metabolism such as cell growth or pH changes.

For compounds of interest in the modulation of sickle cell disease, the inhibition by test compounds of an erythrocyte Gardos channel can be assayed using human red blood cells. The degree of inhibition can be measured using a detectable material such as ⁸⁶Rb. In an exemplary assay, utilizing ⁸⁶Rb, Gardos channel inhibition can be assayed by exposing red blood cells to ⁸⁶Rb and a test compound and measuring the amount of ⁸⁶Rb taken up by the cells. Numerous variations on this assay will be apparent to those of skill in the art.

The potency of the compounds of the invention can be assayed using erythrocytes by a method such as that disclosed by Brugnara et al., J. Clin. Invest., 92: 520-526 (1993); and Brugnara et al., J. Biol. Chem., 268(12): 8760-8768 (1993). Utilizing the methods described in these references, both the percent inhibition of the Gardos channel and the IC₅₀ of the compounds of the invention can be assayed. Briefly, erythrocytes are exposed to a test compound and a ⁸⁶Rb-containing medium. The initial rate of ⁸⁶Rb transport can be calculated from a parameter such as the linear least square slope of ⁸⁶Rb uptake by the cell(s). Inhibitory constants can be calculated by standard methods using computer-assisted nonlinear curve fitting.

When used to modulate intraocular pressure, the activity of a IK1 channel blocker compound formed from the hydrolysis of the corresponding prodrug compound of the invention towards an intermediate conductance, calcium activated potassium channel can be assessed using a variety of in vitro and in vivo assays. In one embodiment, the in vivo assays conducted in mammals and disclosed herein, e.g., the rabbit assay in the examples section, are used to identify prodrugs that form intermediate conductance, calcium activated potassium channel blockers for treatment of increased intraocular pressure. In another embodiment, the in vitro assays described herein are used, e.g., radiolabeled rubidium flux. Such assays are used to test for prodrugs that can form inhibitors of intermediate conductance, calcium activated potassium channels and for the identification of compounds that reduce intraocular pressure in a subject. Assays for modulatory compounds include, e.g., measuring current; measuring membrane potential; measure ion flux; e.g., potassium or rubidium; measuring potassium concentration; measuring second messengers and transcription levels; using potassium-dependent yeast growth assays; measuring intraocular pressure, e.g., by administering a compound able to decrease ion flow through intermediate conductance, calcium activated potassium channels to a subject and measuring changes in intraocular pressure.

Other methods for assaying the activity of ion channels and the activity of agents that affect the ion channels are known in the art. The selection of an appropriate assay methods is well within the capabilities of those of skill in the art who. See, for example, Hille, B., IONIC CHANNELS OF EXCITABLE MEMBRANES, Sinaner Associates, Inc. Sunderland, Mass. (1992).

II. Pharmaceutical Compositions of Intermediate Conductance Calcium Activated Potassium Channel Blockers

In another aspect, the present invention provides pharmaceutical compositions comprising a pharmaceutically acceptable excipient and a prodrug compound of the invention.

Formulation of the Compounds (Compositions)

The prodrug compounds of the present invention can be prepared and administered in a wide variety of oral, parenteral and topical dosage forms. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. Accordingly, the present invention also provides pharmaceutical compositions comprising a pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Any method of administering drugs directly to a mammalian eye may be employed to administer, in accordance with the present invention, the compound or compounds to the eye to be treated. The primary effect on the mammal resulting from the direct administration of the compound or compounds to the mammal's eye is a reduction in intraocular pressure. More preferably, one or more intermediate conductance, calcium activated potassium channel blockers and/or additional compounds known to reduce intraocular pressure are applied topically to the eye or are injected directly into the eye. Particularly useful results are obtained when the compound or compounds are applied topically to the eye in an ophthalmic preparation, e.g., as ocular solutions, suspensions, gels or creams, as examples of topical ophthalmic preparations used for dose delivery.

In accordance with the invention the compounds are typically administered in an ophthalmically acceptable carrier in sufficient concentration so as to deliver an effective amount of the compound or compounds to the eye. The compounds are administered in accordance with the present invention to the eye, typically admixed with an ophthalmically acceptable carrier, and optionally with another compound suitable for treatment of glaucoma and/or reduction of intraocular pressure. Any suitable, e.g., conventional, ophthalmically acceptable carrier may be employed including water (distilled or deionized water), saline and other aqueous media, with or without solubility enhancers such as any of the ophthalmically acceptable beta-cyclodextrins. The compounds may be soluble in the carrier which is employed for their administration, so that the compounds are administered to the eye in the form of a solution. Alternatively, a suspension of the compound or compounds (or salts thereof) in a suitable carrier may also be employed.

When forming compositions for topical administration, the compounds are generally formulated as between about 0.001% to 10% w/v, more preferably between about 0.1% to 5% w/v. In one embodiment, the formulation is 1.0% w/v. In one embodiment, the formulations are solutions in water at a pH preferably between about 5.0 to 8.0 pH, preferably pH 7.4±0.3. In another aspect of the invention, the compounds are formulated as suspensions. In a preferred embodiment, the formulation is in a 1% w/v ophthalmic suspension: 1.0% compound of formula V, micronized; 0.06% carbomer (carbopol 1382), NF; 1.0% poloxamer 188, NF; 2.5% glycerin, USP; 0.01% benzalkonium chloride, NF; sodium hydroxide, NF, q.s. pH 7.4±0.3; and purified water, USP (the formulation may be prepared as % w/w for convenience, and higher grades of water, USP, may be substituted). Various preservatives may be used in an ophthalmic preparation. Preservatives include, but are not limited to, benzalkonium chloride, chlorobutanol, thimerosal, phenylmercuric acetate, and phenylmercuric nitrate. Likewise, various vehicles may be used in such ophthalmic preparation. These vehicles include, but are not limited to, polyvinyl alcohol, povidone, cyclodextrins, hydroxypropyl methyl cellulose, poloxamers, carboxymethyl cellulose and hydroxyethyl cellulose. Such preservatives, if utilized, will typically be employed in an amount between about 0.001 and about 1.0 wt %.

Tonicity adjusters may be added as needed or convenient. They include, but are not limited to, salts, particularly sodium chloride, potassium chloride etc., mannitol and glycerin, or any other suitable ophthalmically acceptable tonicity adjuster. Such agents, if utilized, will typically be employed in an amount between about 0.1 and about 10.0 wt %.

Various buffers and means for adjusting pH may be used so long as the resulting preparation is ophthalmically acceptable. Accordingly, buffers include but are not limited to, acetate buffers, titrate buffers, phosphate buffers, and borate buffers. Acids or bases may be used to adjust the pH of these formulations as needed.

In a similar vein, ophthalmically acceptable antioxidants include, but are not limited to, sodium metabisulfite, sodium thiosulfate, acetylcysteine, butylated hydroxyanisole, and butylated hydroxytoluene.

Some compounds may have limited solubility in water and therefore may require a surfactant or other appropriate co-solvent in the composition. Such co-solvents include: Polysorbate 20, 60 and 80; Pluronic F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to increase ocular absorption of the compound, to decrease variability in dispensing the formulations, to decrease physical separation of components of a suspension or emulsion of formulation and/or otherwise to improve the ophthalmic formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The ophthalmic solution (ocular drops) may be administered to the mammalian eye as often as necessary to maintain an acceptable level of intraocular pressure in the eye. In other words, the ophthalmic solution (or other formulation) is administered to the mammalian eye as often as necessary to maintain the beneficial effect of the active ingredient in the eye. Those skilled in the art will recognize that the frequency of administration depends on the precise nature of the active ingredient and its concentration in the ophthalmic formulation. Within these guidelines it is contemplated that the ophthalmic formulation of the present invention will be administered to the mammalian eye once daily. The formulations may be administered to the mammalian eye anywhere from about 1-4× daily, or as otherwise deemed appropriate by the attending physician. The formulations may also be administered in combination with one or more other pharmaceutical compositions known to reduce intraocular pressure in a subject or otherwise have a beneficial effect in a subject, including miotics (e.g., pilocarpine, carbachol, and acetylcholinesterase inhibitors); sympathomimetics (e.g., epinephrine and dipivalylepinephrine); beta-blockers (e.g., betaxolol, levobunolol and timolol); alpha-2 agonists (e.g., para-amino clonidine); carbonic anhydrase inhibitors (e.g., acetazolamide, methazolamide and ethoxzolamide); and prostaglandins and their analogs and derivatives (e.g., latanaprost).

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference.

As will likewise be appreciated by those skilled in the art, the compositions may be formulated in various dosage forms suitable for topical ophthalmic delivery, as described above, including solutions, suspensions, emulsions, gels, and erodible solid ocular inserts. The compositions are preferably aqueous suspensions or solutions. Further, such formulated compositions may also include one or more additional active ingredients in a single vial for delivery to the patient. That is to say, in addition to one or more potassium channel inhibitors present in a single formulation, the present invention additionally contemplates the presence of one or more of the following therewith: miotics (e.g., pilocarpine, carbachol, and acetylcholinesterase inhibitors); sympathomimetics (e.g., epinephrine and dipivalylepinephrine); beta-blockers (e.g., betaxolol, levobunolol and timolol); alpha-2 agonists (e.g., para-amino clonidine); carbonic anhydrase inhibitors (e.g., acetazolamide, methazolamide and ethoxzolamide); and prostaglandins and their analogs and derivatives (e.g., latanaprost) in a single formulation for administration. One skilled in the art will recognize due care will need to be given in selecting such agents for co-administration from a single formulation with due regard for chemical stability and compatibility with other agents (whether active therapeutic agents or excipients) in the composition made available to the patient.

Effective Dosages

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. For example, when administered in methods to reduce sickle cell dehydration and/or delay the occurrence of erythrocyte sickling or distortion in situ, such compositions will contain an amount of active ingredient effective to achieve this result. Similarly, when the pharmaceutical composition is used to treat or prevent glaucoma, a therapeutically effective amount will reduce intraocular pressure below a predetermined pressure threshold. Determination of a therapeutically effective amount of a compound of the invention is well within the capabilities of those skilled in the art, especially in light of the detailed disclosure herein. The prodrug compounds of the present invention may be administered with additional agents to treat a particular condition, such as glaucoma and/or a sickle cell disease.

For any compound described herein, the therapeutically effective amount can be initially determined from cell culture assays. Target concentrations will be those concentrations of active compound(s) that are capable of inducing inhibition of the intermediate conductance, calcium activated potassium channel. In preferred embodiments, said channel activity is at least 25% inhibited. Concentrations of active compound(s) that are capable of inducing at least about 50%, 75%, or even 90% or higher inhibition of the ion channel potassium flux are presently preferred. The percentage of inhibition of the intermediate conductance, calcium activated potassium channel in the patient can be monitored to assess the efficacy of the drug concentration achieved, and the dosage can be adjusted upwards or downwards by the medical practitioner to achieve the desired percentage of inhibition.

As is well known in the art, therapeutically effective amounts for use in humans can also be determined from animal models. For example, a dose for humans can be formulated to achieve a concentration that has been found to be effective in animals. A useful animal model for sickle cell disease is the SAD-I mouse model (Trudel et al., EMBO J. 11: 3157-3165 (1991)). The dosage in humans can be adjusted by monitoring Gardos channel inhibition and adjusting the dosage upwards or downwards, as described above.

A therapeutically effective dose can also be determined from human data for compounds which are known to exhibit similar pharmacological activities, such as clotrimazole and other antimycotic agents (see, e.g., Brugnara et al., JPET 273:266272 (1995)); Benzaquen et al., Nature Medicine 1: 534-540 (1995); Brugnara et al., J Clin. Invest. 97(5): 1227-1234 (1996)). The applied dose can be adjusted based on the relative bioavailability and potency of the administered compound as compared with clotrimazole.

Adjusting the dose to achieve maximal efficacy in humans based on the methods described above and other methods as are well-known in the art is well within the capabilities of the ordinarily skilled artisan.

In the case of local administration, the systemic circulating concentration of administered compound will generally not be of particular importance. In such instances, the compound is administered so as to achieve a concentration at the local area effective to achieve the intended result.

By way of example, when a compound of the invention is used in the prophylaxis and/or treatment of sickle cell disease, including both chronic sickle cell episodes and acute sickle cell crisis, a circulating concentration of administered compound of about 0.001 μM to 20 μM is considered to be effective, with about 0.01 μM to 5 μM being preferred.

Patient doses for oral administration of the compounds described herein, which is the preferred mode of administration for prophylaxis and for treatment of chronic sickle cell episodes, typically range from about 0.01 mg/day to about 100 mg/day, more typically from about 0.1 mg/day to about 10 mg/day, and most typically from about 0.50 mg/day to about 5 mg/day. Stated in terms of patient body weight, typical dosages range from about 0.0001 to about 0.150 mg/kg/day, more typically from about 0.001 to about 0.015 mg/kg/day, and most typically from about 0.01 to about 0.10 mg/kg/day.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In one embodiment of the invention, the dosage range is 0.001% to 10% w/v. In another embodiment, the dosage range is 0.1% to 5% w/v. In another embodiment, the dosage range is 10-1000 μg per eye. In another embodiment, the dosage range is 75-150 μg per eye.

For other modes of administration, dosage amount and interval can be adjusted individually to provide levels of the administered compound effective for the particular clinical indication being treated. For example, if acute sickle crises are the most dominant clinical manifestation, in one embodiment, a compound according to the invention can be administered in relatively high concentrations multiple times per day. Alternatively, if the patient exhibits only periodic sickle cell crises on an infrequent, periodic or irregular basis, in one embodiment, it may be more desirable to administer a compound of the invention at minimal effective concentrations and to use a less frequent administration regimen. This will provide a therapeutic regimen that is commensurate with the severity of the individual's sickle cell disease.

Utilizing the teachings provided herein, an effective prophylactic or therapeutic treatment regimen can be planned which does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

Therapeutic Index

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch.1, p.1, 1975. The exact formulation, route of administration and dosage can be chosen by the individual physician in view of the patient's condition and the particular method in which the compound is used.

III. Methods for Decreasing Ion Flow in Intermediate Conductance, Calcium Activated Potassium Channels

In addition to the compounds and pharmaceutical formulations discussed in detail above, the present invention provides a number of methods in which the compounds of the invention find use. The methods include, but are not limited to, those that are used in a laboratory setting to probe the basic mechanisms of intermediate conductance, calcium activated potassium channels and channel-active compounds, e.g., pharmacokinetics, drug activity, disease origin and progression and the like.

Thus, in another aspect, the invention provides a method of inhibiting potassium flux of a cell. The method comprises, contacting a cell with an effective amount of a prodrug compound of the invention.

This aspect of the invention has a wide range of uses, but it is preferred as a modality for the study of the basic mechanisms underlying potassium flux and the mechanism of activity of agents that modulate this flux. Further, the compounds of the invention can be utilized as tools in the discovery of new agents that modulate potassium flux. For example, the compounds of the invention can be utilized in assays, such as competitive assays, to test the efficacy of putative inhibitors of potassium flux. These methods of the invention can be performed both in vitro and in vivo. Assays according to the present invention can be carried out by, for example, modifying art-recognized methods to allow the incorporation of the compounds of the invention into them. Such modification is well within the skill of those of skill in the art.

The methods provided in this aspect of the invention are also useful for the diagnosis of conditions that can be treated by modulating ion flux through intermediate conductance, calcium activated potassium channels, or for determining if a patient will be responsive to therapeutic agents, which act by blocking potassium channels. In particular, a patient's cell sample can be obtained and contacted with a compound of the invention and the ion flux can be measured relative to a cell's ion flux in the absence of a compound of the invention. A decrease in ion flux will typically indicate that the patient will be responsive to a therapeutic regimen of ion channel openers.

IV. Methods for Treating Conditions Mediated by Intermediate Conductance, Calcium Activated Potassium Channels

In another preferred embodiment, this method is used to treat or prevent a condition that can be positively affected by modulating potassium flux. In a presently preferred embodiment, the condition is sickle cell disease or glaucoma.

For example, in sickle cell disease, the invention provides a method for reducing erythrocyte dehydration. This method comprises, contacting an erythrocyte with an effective amount of a compound of the invention. This aspect of the invention can be used for a range of purposes including, for example, study of the mechanism of erythrocyte dehydration, investigation of compounds that inhibit or reverse erythrocyte dehydration and the treatment or prevention of conditions associated with erythrocyte dehydration.

In another aspect, the invention provides a method of treating or preventing sickle cell disease. The method comprises administering to a subject suffering sickle cell disease a therapeutically effective amount of one or more compounds of the invention with or without one or more other agents useful in ameliorating the effects of the disease. This aspect of the invention can be utilized to prevent the onset of acute sickle cell events or to ameliorate the effects of these events. Further, the method can be used to treat and/or prevent chronic sickle cell disease. The method can make use of the compounds of the invention per se or, preferably, the pharmaceutical formulations of the invention. The relevant modes of administration, choice of dosage levels and frequency of dosing are discussed above.

The following examples are provided by way of illustration only and not by way of limitation. Those of skill in the art will readily recognize a variety of non-critical parameters that can be changed or modified to yield similar results.

### EXAMPLES

### Example 1

### Exemplary Synthesis of a Sulfone-Containing Compound

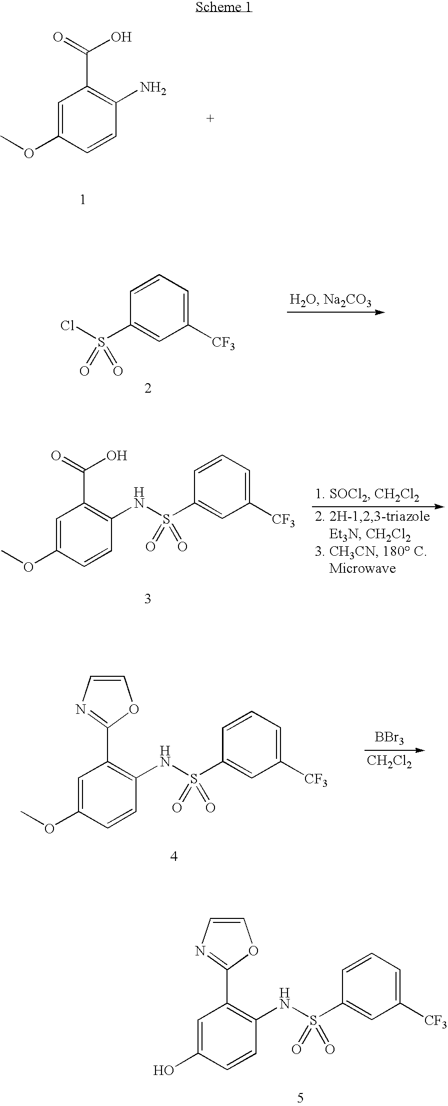

1.1 Synthesis of 3

A suspension of 1 (30 mmol) in 100 mL of sat. Na₂CO₃ was heated at 50° C. for 10 min. After the solution cooled to room temperature, 2 (30 mmol) was added and stirring continued for 2 h. The reaction mixture was acidified with 1 N HCl and extracted with EtOAc (3×100 mL). The combined organic phase was washed with saturated NaCl, dried over MgSO₄, and concentrated under reduced pressure. The residue was purified by column chromatography on silica gel to give 25 mmol of 3.

Analytical data for exemplary compound of the structure 3 is provided below.

5-Methoxy-2-(3-trifluoromethyl-benzenesulfonylamino)-benzoic acid: ¹H NMR (300 MHz, CDCl₃) δ 9.75 (s, 1H), 8.04 (s, 1H), 7.89 (d, 1H), 7.77 (d, 1H), 7.69 (d, 1H), 7.55 (t, 1H), 7.46 (d, 1H), 7.16 (dd, 2H), 3.81 (s, 3H); MS m/z: 376 (M+1).

1.2 Synthesis of 4

To a solution of3 (10 mmol) in 50 mL of anhydrous toluene was added 15 mmol of thionyl chloride and the resulting solution was heated at 80° C. for 2 h. After the solution was cooled to room temperature, the solvent and the excess thionyl chloride were removed under reduced pressure and the residue was further dried under high vacuum for 1 h. To a solution of the residue in 100 mL of dichloromethane and 30 mmol of triethyl amine was added 11 mmol of 2H-1, 2, 3-triazole at 0° C. and the resulting mixture was stirred at room temperature for 2 h. The solvent was removed under reduced pressure and the residue was dissolved in 30 mL of acetonitrile. The mixture was divided into 8 microwave reactor vials (10 mL) and each of the vials was stirred for 40 min at 180° C. in the microwave reactor. The combined mixture was dried under reduced pressure and the residue was purified by column chromatography on silica gel to give 2.25 mmol of 4.

Analytical data for exemplary compound of the structure 4 is provided below.

N-(4-Methoxy-2-oxazol-2-yl-phenyl)-3-trifluoromethyl-benzenesulfonamide: ¹H NMR (300 MHz, CDCl₃) δ 7.96 (s, 1H), 7.79 (d, 1H), 7.72 (d, 1H), 7.63 (d, 1H), 7.62 (s, 1H), 7.42 (d, 1H), 7.31 (d, 1H), 7.24 (s, 1H), 6.97 (dd, 1H), 3.80 (s, 3H); MS m/z: 399 (M+1).

1.3 Synthesis of 5

A solution of 0.75 mmol of 4 and 8 mmol of BBr₃ in 25 mL of dichloromethane was stirred for 4 h at room temperature. The reaction was quenched slowly with sat. NaHCO₃ and the mixture was extracted with EtOAc (3×30 mL). The combined organic phase was washed with saturated NaCl, dried over MgSO₄, and concentrated under reduced pressure. The residue was purified by column chromatography on silica gel to give 0.42 mmol of 5.

Analytical data for exemplary compound of the structure 5 is provided below.

N-(4-Hydroxy-2-oxazol-2-yl-phenyl)-3-trifluoromethyl-benzenesulfonamide: ¹H NMR (300 MHz, CDCl₃) δ 10.90 (s, 1H), 7.96 (s, 1H), 7.79 (d, 1H), 7.73-7.55 (m, 3H), 7.41 (t, 1H), 7.30 (d, 1H), 7.24 (d, 1H), 6.91 (dd, 1H), 3.80 (s, 3H); MS m/z: 385 (M+1).

### Example 2

### Synthesis of a Sulfone-Containing Phosphate Prodrug

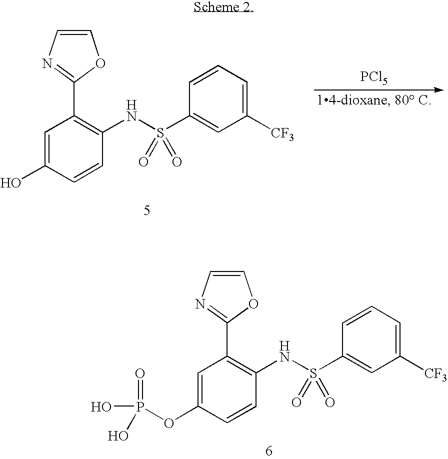

A suspension of 5 (0.16 mmol) and PCl₅ (0.32 mmol) in anhydrous 1,4-dioxane (20 mL) was stirred for 4 h at 80° C. After the solution was cooled to room temperature, the reaction was quenched with sat. NaHCO₃ and the aqueous mixture was stirred for 20 min before it was acidified with 1 N HCl. The acidic mixture was extracted with EtOAc (5×20 mL). The combined organic phase was dried over MgSO₄ and concentrated under reduced pressure. The residue was purified by column chromatography on silica gel to give 0.04 mmol of 6.

Analytical data for exemplary compound of the structure 6 is provided below.

Phosphoric acid mono-[3-oxazol-2-yl-4-(3-trifluoromethyl-benzenesulfonylamino)-phenyl]ester: ¹H NMR (300 MHz, CD₃OD) δ 7.91 (bs, 2H), 7.82 (d, 1H), 7.78-7.75 (m, 2H), 7.69 (d, 1H), 7.55 (t, 1H), 7.35-7.32 (m, 2H); MS m/z: 465 (M+1).

### Example 3

### Synthesis of a Sulfone-Containing Carbonylamine Prodrug

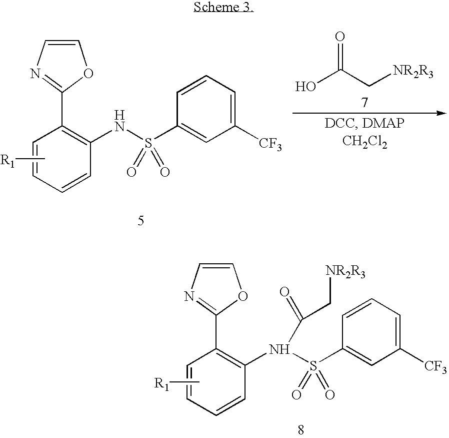

A solution of 5 (0.37 mmol), 1.3-dicyclohexylcarbodiimide (DCC) (0.55 mmol), 7 (0.55 mmol), and 4-(dimethylamino)pyridine (DMAP) (0.02 mmol) in anhydrous dichloromethane (15 mL) was stirred for 2 days at room temperature. The solvent was removed under reduced pressure and the residue was purified by column chromatography on silica gel to give 0.13 mmol of 8. 8 was converted to an HCl salt with HCl in 1.4-dioxane or in water.

N-[4,5-Dimethoxy-2-(3-methyl-[1,2,4]oxadiazol-5-yl)-phenyl]-N-(2-dimethylamino-acetyl)-3-trifluoromethyl-benzenesulfonamide HCl salt: ¹H NMR (300 MHz, CD₃OD) δ 10.19 (bs, 1H), 8.19 (d, 1H), 8.07 (d, 1H), 8.06 (s, 1H), 7.85 (t, 1H), 7.63 (s, 1H), 7.32 (s, 1H), 4.25 (d, 1H), 3.95 (s, 3H), 3.37 (s, 3H), 2.65 (s, 3H), 2.48 (s, 6H); MS m/z: 529 (M+1).

TABLE 1
Representative compounds of the invention

| Number | NAME |
|---|---|
| 1 | Phosphoric acid mono-[3-oxazol-2-yl-4-(3-trifluoromethyl- |
| | benzenesulfonylamino)-phenyl]ester |
| 2 | Amino-acetic acid 3-oxazol-2-yl-4-(3-trifluoromethyl- |
| | benzenesulfonylamino)-phenyl ester |
| 3 | N-(2-Amino-acetyl)-N-[4-fluoro-2-(3-methyl- |
| | [1,2,4]oxadiazol-5-yl)-phenyl]-3-trifluoromethyl- |
| | benzenesulfonamide |
| 4 | N-[4,5-Dimethoxy-2-(3-methyl-[1,2,4]oxadiazol- |
| | 5-yl)-phenyl]-N-(2-dimethylamino-acetyl)-3- |
| | trifluoromethyl-benzenesulfonamide |
| 5 | N-(2-Amino-acetyl)-N-[5-methoxy-2-(3-methyl- |
| | [1,2,4]oxadiazol-5-yl)-phenyl]-3-trifluoromethyl- |
| | benzenesulfonamide |
| 6 | N-(2-Amino-acetyl)-N-(5-fluoro-2-oxazol-2-yl-phenyl)-3- |
| | trifluoromethyl-benzenesulfonamide |
| 7 | N-(2-Amino-acetyl)-N-[4,5-dimethoxy-2-(3-methyl- |
| | [1,2,4]oxadiazol-5-yl)-phenyl]-3-trifluoromethyl- |
| | benzenesulfonamide |
| 8 | Phosphoric acid mono-[4-(3-methyl-[1,2,4]oxadiazol-5- |
| | yl)-3-(3-trifluoromethyl-benzenesulfonylamino)-phenyl]ester |
| 9 | N-(2-Amino-acetyl)-N-(4-methoxy-2-oxazol-2-yl-phenyl)-3- |
| | trifluoromethyl-benzenesulfonamide |
| 10 | Amino-acetic acid 4-[(2-amino-acetyl)-(3-trifluoromethyl- |
| | benzenesulfonyl)-amino]-3-oxazol-2-yl-phenyl ester |
| 11 | N-(2-Amino-acetyl)-N-[4,5-difluoro-2-(3-methyl- |
| | [1,2,4]oxadiazol-5-yl)-phenyl]-3-trifluoromethyl- |
| | benzenesulfonamide |
| 12 | N-(2-Amino-acetyl)-N-(4-methoxy-2-oxazol-2-yl-phenyl)-3- |
| | trifluoromethyl-benzenesulfonamide |
| 13 | N-(2-Dimethylamino-acetyl)-N-(4-methoxy-2-thiazol-2-yl- |
| | phenyl)-3-trifluoromethyl-benzenesulfonamide |

### Example 4

### In Vitro Conversion of a Prodrug to Active IK1 Blocker

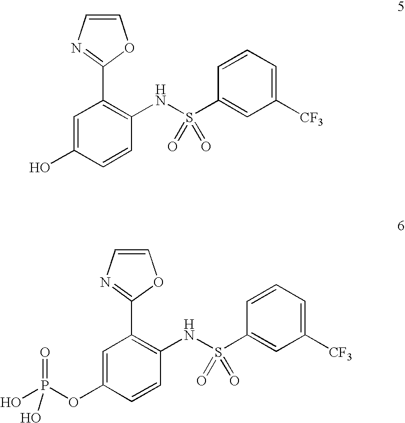

Enzymatic Procedure: 100 μL of 2 mg/mL of alkaline phosphatase or 100 μL of amidase was added according to procedures well known in the art for liver microsome assays. Potassium phosphate buffer and NADPH regenerating solutions were included. The reactions were heated and gently stirred in a 37° C. water bath. Reactions were arrested at appropriate time points by addition of acetonitrile. Samples were analyzed using LC/MS-MS with a 4.2 min gradient.

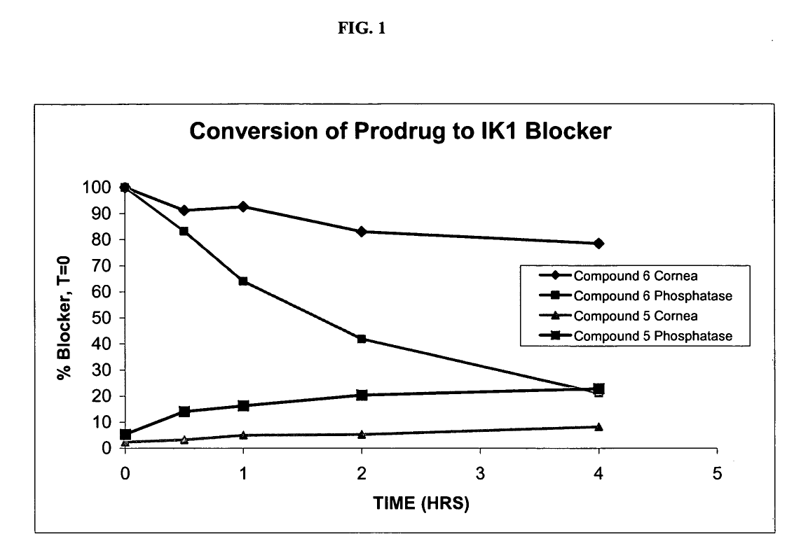

Cornea Homogenate Procedure: 4% cornea homogenate (approximately 40 μL) was added according to procedures well known in the art for liver microsome assays. This includes a potassium phosphate buffer and NADPH regenerating solutions. The reactions were heated and gently stirred in a 37° C. water bath. Reactions were arrested at appropriate time points by addition of Acetonitrile. Samples were analyzed using LC/MS-MS with a 4.2 min gradient. Results are shown in FIG. 1.

### Example 5

### Prodrug Ocular Pharmacokinetic Studies of a Prodrug in Rabbit Eyes

50 microliters of a 0.1% solution of 6 was topically administered to the rabbit eye (n=3). The aqueous humor, iris-ciliary body, and retinal tissue were collected at 1 hr and 3 hrs and analyzed.

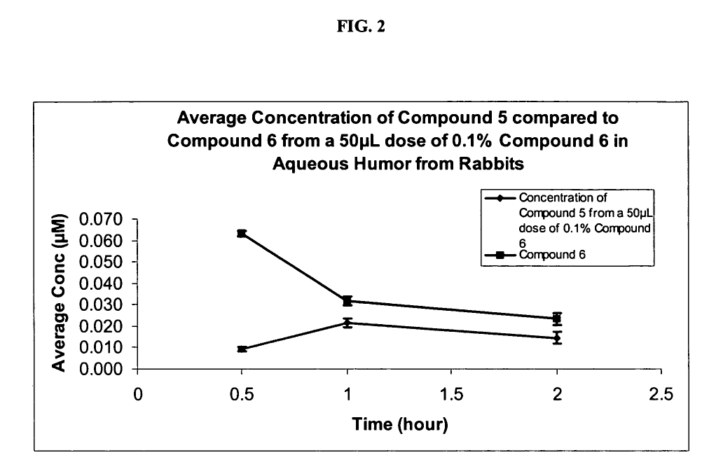

The results are shown in FIG. 2.

### Example 6

### Reduction of Intraocular Pressure in Rabbit Eyes using a Sulfone-Containing Prodrugs

Normotensive rabbits were maintained on a reversed 12 hour light: 12 hour dark light cycle. Testing was performed during the dark phase when natural diurnal rhythms produce elevated pressures (20-25 mm Hg). Applanation tonometry (Model 30 Medtronic pneumatonometer) was used to determine IOP under topical tetracaine anesthesia. Compound 6 was administered topically to the eyes of pigmented rabbits in saline or other suitable (pH 5.0-8.4) aqueous vehicle (50 microliters per eye). Intraocular pressure was determined by applanation tonometry at the time of dosing and at hourly intervals thereafter up to five hours. Pupil diameter was also measured at each pressure reading. Visine° Tears was used as the vehicle (which contains the following ingredients: active ingredients are glycerin (0.2%), hypromellose (0.2%) polyethylene glycol 400 (1%); inactive ingredients are ascorbic acid, dextrose, disodium phosphate, glycine, magnesium chloride, potassium chloride, purified water, sodium chloride, sodium citrate, sodium lactate, and sodium phosphate.

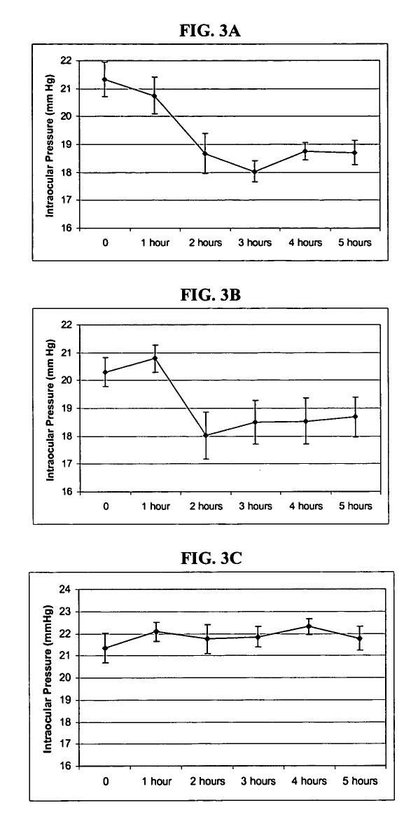

Results are shown in FIGS. 3A, 3B and 3C.

1. A compound having the structure: 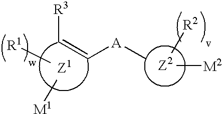
wherein
w and v are integers independently selected from 0 to 4;
ring system Z¹ is a member selected from aryl, and 5-membered heterocycloalkyl;
ring system Z² is a member selected from aryl, heteroaryl, (C₅-C₇)cycloalkyl, and 5 to 7 membered heterocycloalkyl;
R¹ and R² are members independently selected from —NH₂, —NO₂, —CF₃, —OCF₃, hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a substituted or unsubstituted fused ring cycloalkyl, heterocycloalkyl, aryl, or heteroaryl ring, and —OR⁹, wherein
R⁹ is a member selected from hydrogen, —CF₃, substituted or unsubstituted alkyl and substituted or unsubstituted heteroalkyl;
R³ is a member selected from —C(O)OR^{3A}, 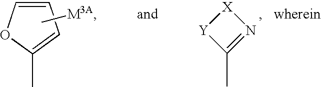
R^{3A} is a member selected from methyl, ethyl and —CF₃,
X is a member selected from —N═N—, —N═C(R^{3B})—, —C(R^{3B}R^{3C})—C(R^{3D}R^{3E})— and —C(R^{3B})═C(R^{3C}), wherein
R^{3B}, R^{3C}, R^{3D}, and R^{3E} are members independently selected from hydrogen, substituted and unsubstituted lower alkyl, -M^{3B} and —CF₃,
Y is a member selected from —O—, —N(M^{3C})— and —S—, and
A is a member selected from -L¹-N(M⁴)-S(O)₂-L²-, and -L¹-S(O)_{q}-L²-, wherein
q is an integer selected from 0 to 2, and
L¹ and L² are members independently selected from a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene;
M¹, M², M^{3A}, and M^{3B} are members independently selected from hydrogen and -L⁵-X⁵—R⁵, wherein
L⁵ is a member independently selected from a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene,
X⁵ is a member independently selected from —O— and —N(R⁶)—, wherein
R⁶ is a member selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, and
R⁵ is a member independently selected from —C(O)—R^{5A} and 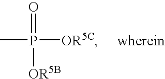
R^{5B} and R^{5C} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, and
R^{5A} is a independently member independently selected from -L^{5A1}-NR^{5A1}R^{5A2} and —OR^{5A3}, wherein
L^{5A1} is a member independently selected from a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene,
R^{5A1} and R^{5A3} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, and
R^{5A2} is a member independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, and —C(O)R^{5A5}, wherein
R^{5A5} is a member independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl; and
M^{3C} and M⁴ are members independently selected from hydrogen, —C(O)—R^{7A}, and 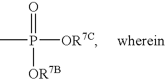
R^{7B} and R^{7C} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, and
R^{7A} is a member independently selected from -L^{7A1}-NR^{7A1}R^{7A2}, and —OR^{7A3}, wherein
L^{7A1} is a member independently selected from a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene,
R^{7A1} and R^{7A3} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, and
R^{7A2} is a member independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, and —C(O)R^{7A5}, wherein
R^{7A5} is a member independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl;
wherein at least one of M¹, M², M^{3A}, M^{3B}, M^{3C}, and M⁴ is not hydrogen.

2. The compound of claim 1, wherein w is selected from 0 to 3.

3. The compound of claim 1, wherein w is 1 and v is 1.

4. The compound of claim 1, wherein w is 0 and v is 1.

5. The compound of claim 1, wherein A is a member selected from —N(M⁴)-S(O)₂—, -L¹-S(O)_{q}—, and —S(O)q-L²-, wherein
q is an integer selected from 0 to 2, and
L¹ and L² are substituted or unsubstituted alkylene.

6. The compound of claim 5, wherein L¹ and L² are substituted or unsubstituted (C₁-C₅)alkylene.

7. The compound of claim 5, wherein L¹ and L² are —C(R^{8A}R^{8B})—, wherein R^{8A} and R^{8B} are members independently selected from hydrogen, substituted and unsubstituted lower alkyl, —OR^{4C} and —CF₃, wherein
R^{4C} is a member selected from hydrogen, and substituted or unsubstituted lower alkyl.

8. The compound of claim 1, wherein A is —N(M⁴)-S(O)₂—.

9. The compound of claim 8, wherein M⁴ is —C(O)—R^{7A}.

10. The compound of claim 9, wherein M¹, M², M^{3A}, M^{3B}, and M^{3C} are hydrogen.

11. The compound of claim 1, wherein A is —N(H)—S(O)₂—.

12. The compound of claim 1, wherein Z¹ is a member selected from phenyl and thiophenyl.

13. The compound of claim 1, wherein R³ is a member selected from 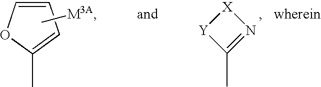
X is a member selected from —N═C(R^{3B})—, —C(R^{3B}R^{3C})—, —C(R^{3D}R^{3E})— and —C(R^{3B})═C(R^{3C})—, and
Y is a member selected from the group consisting of O and S.

14. The compound of claim 1, wherein Z² is a member selected from the group consisting of: 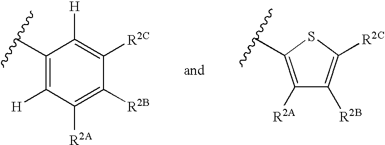
wherein
R^{2A}, R^{2B} and R^{2C} are members independently selected from halogen, lower alkyl, —OR^{2D}, —CF₃, NO₂, Cl, and M²; wherein
R^{2D} is a member selected from the group consisting of H, lower alkyl, substituted lower alkyl, and —CF₃, and
M² is -L⁵-X⁵—R⁵, wherein L⁵ is a bond and X⁵ is —O—.

15. The compound of claim 14, wherein Z² is:

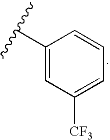

16. The compound of claim 1, wherein
M¹, M², M^{3A}, and M^{3B} are members independently selected from hydrogen and -L⁵-X⁵—R⁵, wherein
L⁵ is a member independently selected from a bond, substituted or unsubstituted (C₁-C₁₀) alkylene, and substituted or unsubstituted 2 to 10 membered heteroalkylene,
X⁵ is a member independently selected from —O— and —N(R⁶)—, wherein
R⁶ is a member independently selected from hydrogen, substituted or unsubstituted (C₁-C₁₀) alkyl, and substituted or unsubstituted 2 to 10 membered heteroalkyl, and
R⁵ is a member independently selected from —C(O)—R^{5A} and 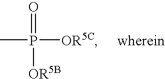
R^{5B} and R^{5C} are members independently selected from hydrogen, substituted or unsubstituted (C₁-C₁₀) alkyl, and substituted or unsubstituted 2 to 10 membered heteroalkyl, and
R^{5A} is a member independently selected from -L^{5A1}-NR^{5A1}R^{5A2} and OR^{5A3}, wherein
L^{5A1} is a member independently selected from substituted or unsubstituted (C₁-C₁₀) alkylene, and substituted or unsubstituted 2 to 10 membered heteroalkylene,
R^{5A1} and R^{5A3} are members independently selected from hydrogen, substituted or unsubstituted (C₁-C₁₀) alkyl, and substituted or unsubstituted 2 to 10 membered heteroalkyl, and
R^{5A2} is a member independently selected from hydrogen, substituted or unsubstituted (C₁-C₁₀) alkyl, and substituted or unsubstituted 2 to 10 membered heteroalkyl, and —C(O)R^{5A5}, wherein
R^{5A5} is a member independently selected from hydrogen, substituted or unsubstituted (C₁-C₁₀) alkyl, and substituted or unsubstituted 2 to 10 membered heteroalkyl; and
M^{3C} and M⁴ are members independently selected from hydrogen, —C(O)—R^{7A} and 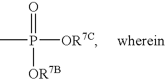
R^{7B} and R^{7C} are members independently selected from hydrogen, substituted or unsubstituted (C₁-C₁₀) alkyl, and substituted or unsubstituted 2 to 10 membered heteroalkyl, and
R^{7A} is a member independently selected from -L^{7A1}-NR^{7A1}R^{7A2}, and OR^{7A3}, wherein
L^{7A1} is a member independently selected from substituted or unsubstituted (C₁-C₁₀) alkylene, and substituted or unsubstituted 2 to 10 membered heteroalkylene,
R^{7A1} and R^{7A3} are members independently selected from hydrogen, substituted or unsubstituted (C₁-C₁₀) alkyl, and substituted or unsubstituted 2 to 10 membered heteroalkyl, and
R^{7A2} is a member independently selected from hydrogen, substituted or unsubstituted (C₁-C₁₀) alkyl, and substituted or unsubstituted 2 to 10 membered heteroalkyl, and —C(O)R^{7A5}, wherein
R^{7A5} is a member independently selected from hydrogen, substituted or unsubstituted (C₁-C₁₀) alkyl, and substituted or unsubstituted 2 to 10 membered heteroalkyl.

17. The compound of claim 1, wherein
M¹, M², M^{3A}, and M^{3B} are members independently selected from hydrogen and -L⁵-X⁵—R⁵, wherein
L⁵ is a member independently selected from a bond, unsubstituted (C₁-C₁₀) alkylene, and unsubstituted 2 to 10 membered heteroalkylene,
X⁵ is a member independently selected from —O— and —N(R⁶)—, wherein
R⁶ is a member independently selected from hydrogen, unsubstituted (C₁-C₁₀) alkyl, and unsubstituted 2 to 10 membered heteroalkyl, and
R⁵is a member independently selected from —C(O)—R^{5A} and 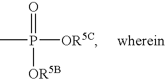
R^{5B} and R^{5C} are members independently selected from hydrogen, unsubstituted (C₁-C₁₀) alkyl, and unsubstituted 2 to 10 membered heteroalkyl, and
R^{5A} is a member independently selected from -L^{5A1}-NR^{5A1}R^{5A2} and OR^{5A3}, wherein
L^{5A1} is a member selected from unsubstituted (C₁-C₁₀) alkylene, and unsubstituted 2 to 10 membered heteroalkylene,
R^{5A 1} and R^{5A3} are members independently selected from hydrogen, unsubstituted (C₁-C₁₀) alkyl, and unsubstituted 2 to 10 membered heteroalkyl, and
R^{5A2} is a member independently selected from hydrogen, unsubstituted (C₁-C₁₀) alkyl, and unsubstituted 2 to 10 membered heteroalkyl, and —C(O)R^{5A5}, wherein
R^{5A5} is a member independently selected from hydrogen, unsubstituted (C₁-C₁₀) alkyl, and unsubstituted 2 to 10 membered heteroalkyl; and
M^{3C} and M⁴ are members independently selected from hydrogen, —C(O)—R^{7A} and 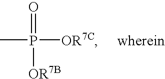
wherein
R^{7B} and R^{7C} are members independently selected from hydrogen, unsubstituted (C₁-C₁₀) alkyl, and unsubstituted 2 to 10 membered heteroalkyl, and
R^{7A} is a member independently elected from -L^{7A1}-NR^{7A1}R^{7A2}, and OR^{7A3}, wherein
L^{7A1} is a member independently selected from unsubstituted (C₁-C₁₀) alkylene, and unsubstituted 2 to 10 membered heteroalkylene,
R^{7A1} and R^{7A3} are members independently selected from hydrogen, unsubstituted (C₁-C₁₀) alkyl, and unsubstituted 2 to 10 membered heteroalkyl, and
R^{7A2} is a member independently selected from hydrogen, unsubstituted (C₁-C₁₀) alkyl, and unsubstituted 2 to 10 membered heteroalkyl, and —C(O)R^{7A5}, wherein
R^{7A5} is a member independently selected from hydrogen, unsubstituted (C₁-C₁₀) alkyl, and unsubstituted 2 to 10 membered heteroalkyl.

18. The compound of claim 1, wherein
M¹, M², M^{3A}, and M^{3B} are members independently selected from hydrogen and -L⁵-X⁵—R⁵, wherein ’L⁵ is a bond,
X⁵ is a member independently selected from —O— and —N(R⁶)—, wherein
R⁶ is a member independently selected from hydrogen, unsubstituted (C₁-C₅) alkyl, and unsubstituted 2 to 5 membered heteroalkyl,
R⁵ is a member independently selected from —C(O)—R^{5A} and 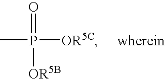
wherein
R^{5B} and R^{5C} are members independently selected from hydrogen, unsubstituted (C₁-C₅) alkyl, and unsubstituted 2 to 5 membered heteroalkyl, and
R^{5A} is a member independently selected from -L^{5A1}-NR^{5A1}R^{5A2} and OR^{5A3}, wherein
L^{5A1} is unsubstituted (C₁-C₅) alkylene,
R^{5A1} and R^{5A3} are members independently selected from hydrogen, unsubstituted (C₁-C₅) alkyl, and unsubstituted 2 to 5 membered heteroalkyl, and
R^{5A2} is a member independently selected from hydrogen, unsubstituted (C₁-C₅) alkyl, unsubstituted 2 to 5 membered heteroalkyl, and —C(O)R^{5A5}, wherein
R^{5A5} is a member independently selected from hydrogen, unsubstituted (C₁-C₅) alkyl, and unsubstituted 2 to 5 membered heteroalkyl; and
M^{3C} and M⁴ are members independently selected from hydrogen, —C(O)—R^{7A} and 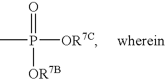
wherein
R^{7B} and R^{7C} are members independently selected from hydrogen, unsubstituted (C₁-C₅) alkyl, and unsubstituted 2 to 5 membered heteroalkyl, and
R^{7A} is a member independently selected from -L^{7A1}-NR^{7A1}R^{7A2}, and OR^{7A3}, wherein
L^{7A1} is a member independently selected from unsubstituted (C₁-C₅) alkylene,
R^{7A1} and R^{7A3} are members independently selected from hydrogen, unsubstituted (C₁-C₅) alkyl, and unsubstituted 2 to 5 membered heteroalkyl, and
R^{7A2} is a member independently selected from hydrogen, unsubstituted (C₁-C₅) alkyl, and unsubstituted 2 to 5 membered heteroalkyl, and —C(O)R^{7A5}, wherein
R^{7A5} is a member independently selected from hydrogen, unsubstituted (C₁-C₅) alkyl, and unsubstituted 2 to 5 membered heteroalkyl.

19. The compound of claim 1, wherein
M¹, M², M^{3A}, and M^{3B} are members independently selected from hydrogen and -L⁵-X⁵—R⁵, wherein
L⁵ is a bond,
X⁵ is —O—,
R⁵ is a member independently selected from —C(O)—R^{5A} and 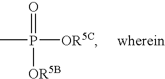
wherein
R^{5B} and R^{5C} are hydrogen, and
R^{5A} is -L^{5A1}-NR^{5A1}R^{5A2}, wherein
L^{5A1} is unsubstituted (C₁-C₅) alkylene, and
R^{5A1} and R^{5A2} are members independently selected from hydrogen, unsubstituted (C₁-C₅) alkyl; and
M^{3C} and M⁴ are members independently selected from hydrogen, and —C(O)—R^{7A}, wherein
R^{7A} is L^{7A1}-NR^{7A1}R^{7A2}, wherein
L^{7A1} is unsubstituted (C₁-C₅) alkylene, and
R^{7A1} and R^{7A2} are members independently selected from hydrogen, unsubstituted (C₁-C₅) alkyl.

20. The compound of claim 8, wherein
M², M^{3A}, M^{3B}, M^{3C}, and M⁴ are hydrogen; and
M¹ is -L⁵-X⁵—R⁵, wherein
L⁵ is a bond,
X⁴ is —O—,
R⁵ is a member independently selected from —C(O)—R^{5A} and 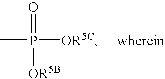
wherein
R^{5B} and R^{5C} are hydrogen, and
R^{5A} is -L^{5A1}-NR^{5A1}R^{5A2}, wherein
L^{5A1} is unsubstituted (C₁-C₅) alkylene, and
R^{5A1} and R^{5A2} are members independently selected from hydrogen, and unsubstituted (C₁-C₅) alkyl.

21. The compound of claim 8, wherein
M¹, M², M^{3A}, M^{3B}, and M^{3C} are hydrogen; and
M⁴ is —C(O)—R^{7A}, wherein
R^{7A} is -L^{7A1}-NR^{7A1}R^{7A2}, wherein
L^{7A1} is unsubstituted (C₁-C₅) alkylene,
R^{7A1} and R^{7A2} are members independently selected from hydrogen, unsubstituted (C₁-C₅) alkyl.

22. The compound of claim 1, wherein
R¹ and R² are members independently selected from —NO₂, —CF₃, hydrogen, halogen, substituted or unsubstituted (C₁-C₁₀)alkyl, substituted or unsubstituted 2 to 10 membered heteroalkyl, substituted or unsubstituted (C₅-C₇) cycloalkyl, substituted or unsubstituted 5 to 7 membered heterocycloalkyl, substituted or unsubstituted aryl, substituted or unsubstituted heteroaryl, a fused ring heterocycloalkyl, and OR⁹, wherein
R⁹ is a member independently selected from —CF₃, substituted or unsubstituted (C₁-C₅)alkyl and substituted or unsubstituted 2 to 5 membered heteroalkyl.

23. The compound of claim 1, wherein
R¹ and R² are members independently selected from —NO₂, —CF₃, hydrogen, halogen, unsubstituted (C₁-C₁₀)alkyl, unsubstituted 2 to 10 membered heteroalkyl, unsubstituted (C₅-C₇) cycloalkyl, unsubstituted 5 to 7 membered heterocycloalkyl, unsubstituted aryl, unsubstituted heteroaryl, OR⁹ and —C(O)OR¹⁰, wherein
R¹⁰ is unsubstituted (C₁-C₁₀)alkyl, and
R⁹ is a member independently selected from —CF₃, unsubstituted (C₁-C₅)alkyl and unsubstituted 2 to 5 membered heteroalkyl.

24. The compound of claim 1, wherein
R¹ and R² are members independently selected from —NO₂, —CF₃, hydrogen, halogen, unsubstituted (C₁-C₁₀)alkyl, unsubstituted 2 to 10 membered heteroalkyl, OR⁹ and —C(O)OR¹⁰, wherein
R¹⁰ is unsubstituted (C₁-C₅)alkyl, and
R⁹ is a member independently selected from —CF₃, unsubstituted (C₁-C₅)alkyl and unsubstituted 2 to 5 membered heteroalkyl.

25. The compound of claim 1, wherein
R¹ and R² are members independently selected from —NO₂, —CF₃, F, Cl, hydrogen, methyl, ethyl, —C(O)OCH₃, —OCH₃, and —OCF₃.

26. A pharmaceutical formulation comprising a pharmaceutically acceptable excipient and a compound of claim 1.

27. A pharmaceutical formulation comprising a pharmaceutically acceptable excipient and a compound of claim 2.

28. A pharmaceutical formulation comprising a pharmaceutically acceptable excipient and a compound of claim 3.

29. A method of inhibiting potassium flux through intermediate conductance potassium channels in a cell, said method comprising contacting said cell with an effective amount of a compound of claim 1.

30. A method for reducing intraocular pressure in a subject in need thereof by decreasing potassium ion flow through intermediate conductance potassium channels in a cell, the method comprising the step of administering to the subject a therapeutically effective amount compound of claim 1.

31. The method of claim 30, wherein the subject has glaucoma characterized by increased intraocular pressure.

32. The method of claim 30, wherein the method prevents glaucoma characterized by increased intraocular pressure.

33. The method of claim 32, wherein the method further comprises the step of administering to the subject one or more additional agents selected from the group consisting of miotics, beta blockers, alpha-2 agonists, carbonic anhydrase inhibitors, beta adrenergic blockers, prostaglandins and docosanoid are administered to said subject.

34. A method of preventing or retarding dehydration of erythrocytes comprising contacting said erythrocyte with a compound of claim 1.

35. A method for treating or preventing sickle cell disease comprising administering to a subject in need thereof a therapeutically effective amount of a compound of claim 1.

36. The method of claim 35, wherein the method further comprises the step of administering to the subject one or more additional agents selected from the group consisting of hydroxyurea, erythropoietin, and an antibiotic.

37. The method of claim 36, wherein said antibiotic is selected from the group consisting of ceftriaxone and erythromycin.
